(19) United States
(12) Patent Application Publication
(10) Pub. No.: US 2011/0212960 A1
(43) Pub. Date: Sep. 1, 2011

(54) NEW COMPOUNDS FOR THE TREATMENT OF CNS DISORDER

(75) Inventors: Niklas HEINE, Biberach (DE); Cornelia DORNER-CIOSSEK, Warthausen (DE); Christian EICKMEIER, Mittelbiberach (DE); Dennis FIEGEN, Biberach (DE); Thomas FOX, Biberach (DE); Klaus FUCHS, Mittelbiberach (DE); Riccardo GIOVANNINI, Verona (IT); Holger ROSENBROCK, Mittelbiberach (DE); Gerhard SCHAENZLE, Biberach (DE); Lars BAERFACKER, Oberhausen (DE)

(73) Assignee: BOEHRINGER INGELHEIM INTERNATIONAL GMBH, Ingelheim am Rhein (DE)

(21) Appl. No.: 12/855,129

(22) Filed: Aug. 12, 2010

(30) Foreign Application Priority Data
Aug. 12, 2009 (EP) 09 167 716.1

(51) Int. Cl. C07D 487/04; A61K 31/5377; A61K 31/519; A61P 25/28; A61P 25/00; A61P 25/18; A61P 3/10; A61P 3/06; A61P 3/00; A61P 1/00; A61P 9/00; A61P 11/00

(52) U.S. Cl. 514/234.2; 544/118; 514/262.1; 544/262

(57) ABSTRACT

The invention relates to novel pyrazolopyrimidinones according to formula (I).

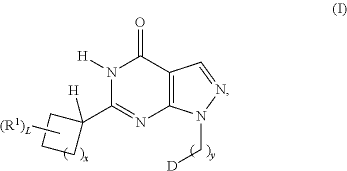

The new compounds shall be used for the manufacture of medicaments, in particular medicaments for the treatment of conditions concerning deficits in perception, concentration, learning or memory. The new compounds are also for the manufacture of medicaments for the treatment of Alzheimer's disease. Further aspects of the present invention refer to a process for the manufacture of the compounds and their use for producing medicaments.

The invention relates to novel pyrazolopyrimidinones. The new compounds shall be used for the manufacture of medicaments, in particular medicaments for the treatment of conditions concerning deficits in perception, concentration, learning or memory. The new compounds are also for the manufacture of medicaments for the treatment of Alzheimer's disease. Further aspects of the present invention refer to a process for the manufacture of the compounds and their use for producing medicaments.

### BACKGROUND OF THE INVENTION

The inhibition of phosphodiesterase 9A (PDE9A) is one of the currents concepts to find new access paths to the treatment of cognitive impairments due to CNS disorders like Alzheimer's Disease or due to any other neurodegenerative process of the brain. With the present invention, new compounds are presented that follow this concept.

Phosphodiesterase 9A is one member of the wide family of phosphodiesterases. These kinds of enzymes modulate the levels of the cyclic nucleotides 5′-3′ cyclic adenosine monophosphate (cAMP) and 5′-3′ cyclic guanosine monophosphate (cGMP). These cyclic nucleotides (cAMP and cGMP) are important second messengers and therefore play a central role in cellular signal transduction cascades. Each of them reactivates inter alia, but not exclusively, protein kinases. The protein kinase activated by cAMP is called protein kinase A (PKA), and the protein kinase activated by cGMP is called protein kinase G (PKG). Activated PKA and PKG are able in turn to phosphorylate a number of cellular effector proteins (e.g. ion channels, G-protein-coupled receptors, structural proteins, transcription factors). It is possible in this way for the second messengers cAMP and cGMP to control a wide variety of physiological processes in a wide variety of organs. However, the cyclic nucleotides are also able to act directly on effector molecules. Thus, it is known, for example, that cGMP is able to act directly on ion channels and thus is able to influence the cellular ion concentration (review in: Wei et al., Prog. Neurobiol., 1998, 56, 37-64). The phosphodiesterases (PDE) are a control mechanism for controlling the activity of cAMP and cGMP and thus in turn for the corresponding physiological processes. PDEs hydrolyse the cyclic monophosphates to the inactive monophosphates AMP and GMP. Currently, 11 PDE families have been defined on the basis of the sequence homology of the corresponding genes. Individual PDE genes within a family are differentiated by letters (e.g. PDE1A and PDE1 B). If different splice variants within a gene also occur, this is then indicated by an additional numbering after the letters (e.g. PDE1A1).

Human PDE9A was cloned and sequenced in 1998. The amino acid identity with other PDEs does not exceed 34% (PDE8A) and is never less than 28% (PDE5A). With a Michaelis-Menten constant (Km) of 170 nanomolar, PDE9A has high affinity for cGMP. In addition, PDE9A is selective for cGMP (Km for cAMP=230 micromolar). PDE9A has no cGMP binding domain, suggesting that the enzyme activity is not regulated by cGMP. It was shown in a Western blot analysis that PDE9A is expressed in humans inter alia in testes, brain, small intestine, skeletal muscle, heart, lung, thymus and spleen. The highest expression was found in the brain, small intestine, kidney, prostate, colon, and spleen (Fisher et al., J. Biol. Chem., 1998, 273 (25), 15559-15564; Wang et al., Gene, 2003, 314, 15-27). The gene for human PDE9A is located on chromosome 21q22.3 and comprises 21 exons. 4 alternative splice variants of PDE9A have been identified (Guipponi et al., Hum. Genet., 1998, 103, 386-392). Classical PDE inhibitors do not inhibit human PDE9A. Thus, IBMX, dipyridamole, SKF94120, rolipram and vinpocetine show no inhibition on the isolated enzyme in concentrations of up to 100 micromolar. An IC50 of 35 micromolar has been demonstrated for zaprinast (Fisher et al., J. Biol. Chem., 1998, 273 (25), 15559-15564).

Murine PDE9A was cloned and sequenced in 1998 by Soderling et al. (J. Biol. Chem., 1998, 273 (19), 15553-15558). This has, like the human form, high affinity for cGMP with a Km of 70 nanomolar. Particularly high expression was found in the mouse kidney, brain, lung and liver. Murine PDE9A is not inhibited by IBMX in concentrations below 200 micromolar either; the 1050 for zaprinast is 29 micromolar (Soderling et al., J. Biol. Chem., 1998, 273 (19), 15553-15558). It has been found that PDE9A is strongly expressed in some regions of the rat brain. These include olfactory bulb, hippocampus, cortex, basal ganglia and basal forebrain (Andreeva et al., J. Neurosci., 2001, 21 (22), 9068-9076). The hippocampus, cortex and basal forebrain in particular play an important role in learning and memory processes. As already mentioned above, PDE9A is distinguished by having particularly high affinity for cGMP. PDE9A is therefore active even at low physiological concentrations, in contrast to PDE2A (Km=10 micromolar; Martins et al., J. Biol. Chem., 1982, 257, 1973-1979), PDE5A (Km=4 micromolar; Francis et al., J. Biol. Chem., 1980, 255, 620-626), PDE6A (Km=17 micromolar; Gillespie and Beavo, J. Biol. Chem., 1988, 263 (17), 8133-8141) and PDE11A (Km=0.52 micromolar; Fawcett et al., Proc. Nat. Acad. Sci., 2000, 97(7), 3702-3707). In contrast to PDE2A (Murashima et al., Biochemistry, 1990, 29, 5285-5292), the catalytic activity of PDE9A is not increased by cGMP because it has no GAF domain (cGMP-binding domain via which the PDE activity is allosterically increased) (Beavo et al., Current Opinion in Cell Biology, 2000, 12, 174-179). PDE9A inhibitors may therefore lead to an increase in the baseline cGMP concentration.

This outline will make it evident that PDE9A engages into specific physiological processes in a characteristic and unique manner, which distinguishes the role of PDE9A characteristically from any of the other PDE family members.

WO04099210 discloses 6-arylmethyl-substituted pyrazolopyrimidinones which are PDE9 inhibitors. The compounds do not have a non-aromatic heterocyclic moiety in the 1 position of the pyrazolopyrimidine.

WO04099211 discloses 6-cyclylmethyl- and 6-alkylmethyl-substituted pyrazolopyrimidines and their use for the improvement of cognition, concentration etc.

DE 102 38 722 discloses the use of PDE9A-inhibitors for the improvement of cognition, concentration etc.

WO04018474 discloses phenyl-substituted pyrazolopyrimidines and their use for the improvement of perception, concentration learning and/or memory.

WO04026876 discloses alkyl-substituted pyrazolopyrimidines which and their use for the improvement of awareness, concentration learning capacity and/or memory performance.

WO04096811 discloses heterocyclic bicycles as PDE9 inhibitors for the treatment of diabetes, including type 1 and type 2 diabetes, hyperglycemia, dyslipidemia, impaired glucose tolerance, metabolic syndrome, and/or cardiovascular disease. Other prior art is directed to chemically similar nucleoside derivatives. As examples it is referred to WO02057425, which discloses nucleoside derivatives, which are inhibitors of RNA-dependent RNA viral polymerase, or WO01060315, which discloses nucleoside derivatives for the treatment of hepatitis C infection or EP679657, which discloses compounds that serve as ribonucleoside analogues or US2002058635, which discloses purine L-nucleoside compounds, in which both the purine rings and the pentose ring are either modified, functionalized, or both. So the pentose ring for example must show at least one esterified hydroxy group.

WO06084281 discloses inhibitors of the E1 activation enzyme that have a sulfonamid moiety.

U.S. Pat. No. 3,732,225 describes pyrazolopyrimidinones which have an antiinflammatory and blood glucose-lowering effect.

DE2408906 describes styrylpyrazolopyrimidinones which can be employed as antimicrobial and anti-inflammatory agents for the treatment of, for example, oedema.

### OBJECTIVE OF THE INVENTION

The above cited prior art makes it evident that changes in the substitution pattern of pyrazolopyrimidinones result in interesting changes concerning biological activity, respectively changes in the affinity towards different target enzymes.

Therefore it is an objective of the present invention to provide compounds that effectively modulate PDE9A for the purpose of the development of a medicament, in particular in view of diseases, the treatment of which is accessible via PDE9A modulation.

It is another objective of the present invention to provide compounds that are useful for the manufacture of a medicament for the treatment of CNS disorders.

Yet another objective of the present invention is to provide compounds which show a favourable side effect profile.

Another objective of the present invention is to provide compounds that have a favourable selectively profile in favour for PDE9A inhibition over other PDE family members and by this may provide advantage.

Yet another objective is to provide such a medicament not only for treatment but also for prevention or modification of the corresponding disease.

### DETAILED DESCRIPTION OF THE PRESENT INVENTION

The compounds of the present invention are characterised by general formula I:

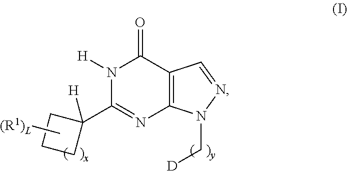

wherein
R¹ is selected independently for each R¹ from the group R^{1a} consisting of Hydrogen, fluorine, chlorine, bromine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, heterocyclyl-CO—, R¹⁰—O—, R¹⁰—O—C₁₋₃-alkyl-, (R¹⁰)₂N—, R¹⁰O—CO—, (R⁹)₂N—CO—, R¹⁰—CO—(R¹⁰)N—, R¹⁰—CO—, (R⁹)₂N—CO—(R¹⁰)N—, (R⁹)₂N—CO—O—, R¹⁰—O—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, heterocyclyl-CO—, and C₁₋₆-alkyl-SO₂— may optionally be substituted independently of one another by one or more substituents selected independently of one another from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, C₃₋₆-cycloalkyl-C₁₋₄-alkyl-, and C₁₋₆-alkyl-, preferably from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹⁰)₂N—C₁₋₃-alkyl-, (R¹⁰)₂N—CO—, C₃₋₆-cycloalkyl-, and C₃₋₆-cycloalkyl-C₁₋₄-alkyl-,
L is selected from the integers 0, 1, 2 and 3,
x is selected from the integers 0, 1, 2, 3 and 4,
y is selected from the integers 0, 1 and 2,
D is selected from the group D^{1a} consisting of heterocyclyl,
   wherein the above-mentioned members of the group D^{1a} may optionally be substituted by one or more substituents selected independently of one another from the group R² and/or optionally substituted by one group R³
      or
D is selected from the group D^{2a} consisting of cyclopropyl, cyclobutyl, cyclopentyl, cyclohexyl, cycloheptyl cyclooctyl, cyclobutenyl, cyclopentenyl, cyclohexenyl, cycloheptenyl, cyclooctenyl, cyclopentadienyl, cyclohexadienyl, cycloheptadienyl, cyclooctadienyl, cycloheptatrienyl, cyclooctatrienyl and cyclooctatetraenyl,
   wherein the above-mentioned members of the group D^{2a} may optionally be substituted by one or more substituents selected independently of one another from the group R⁴,
      or
D is selected from the group D^{3a} consisting of C₁₋₈-alkyl
   wherein the above-mentioned C₁₋₈-alkyl-group D^{3a} may optionally be substituted by one or more substituents selected independently of one another from the group R⁵.
      or
D is selected from the group D^{4a} consisting of aryl
   wherein the above-mentioned aryl group D^{4a} may optionally be substituted by one or more substituents selected independently of one another from the group consisting of R⁶. Preferred are such compounds wherein D^{4a} is substituted by not more than one R⁶.
      or
D is selected from the group D^{5a} consisting of heteroaryl
   wherein the above-mentioned members of the group D^{5a} may optionally be substituted by one or more substituents selected independently of one another from the group R⁶. Preferred are such compounds wherein D^{5a} is substituted by not more than one R⁶.
R² is selected from the group R^{2a} consisting of
   H—, fluorine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, C₁₋₆-alkyl-SO₂—, and oxo,
   where the above-mentioned members HF₂C—, FH₂C—, F₃C—CH₂—, C₁₋₆-alkyl-(Preferably C₂₋₆-alkyl), C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—C₁₋₃-alkyl-, and C₁₋₆-alkyl-SO₂— may optionally be substituted independently of one another by one or more substituents selected independently of one another from the group consisting of
   fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰⁾₂N—, (R¹⁰)₂N—C₁₋₃-alkyl-, and (R¹⁰)₂N—CO—,
   and in cases in that D¹ is a heterocyclyl group with NR² as ring member, R² shall be
      independently of any other R²: H—, F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, R¹⁰O—CO—, (R¹⁰)₂N—CO—, R¹⁰—CO—, R¹⁰—SO₂—, and C₁₋₆-alkyl-SO₂—,
      where the above-mentioned members F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, and C₁₋₆-alkyl-SO₂— may optionally be substituted independently of one another by one or more substituents selected independently of one another from the group consisting of
      fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and (R¹⁰)₂N—CO—,
R³ is selected from the group R^{3a} consisting of
   H—, HO— and R¹⁰—O—,
R⁴ is selected from the group R^{4a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
   or
   two substituents R^{4a} together form a C₂₋₆-alkylene bridge, wherein one or two CH₂ groups of the C₂₋₆-alkylene bridge may be replaced independently of one another by O, S, SO, SO₂, N(R¹⁰) or N—C(O)—R¹⁰ in such a way that in each case two O or S atoms or an O and an S atom are not joined together directly.
      R⁵ is selected from the group R^{5a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂—, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁶ is selected from the group R^{6a} consisting of
   H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
   where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl- and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁹ is selected independently for each R⁹ from the group R^{9a} consisting of H—, F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, and
   where the above-mentioned members F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, may optionally be substituted independently of one another by one or more substituents selected independently of one another from the group consisting of
   fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl-O— and C₁₋₆-alkyl-,
R¹⁰ is selected independently for each R¹⁰ from the group R^{10a} consisting of H— (but not in case is part of a group being selected from R¹⁰O—CO—, R¹⁰—SO₂— or R¹⁰—CO—), F₃C—CH₂—, C₁₋₆-alkyl-, C₂₋₆-alkenyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, and heteroaryl-C₁₋₃-alkyl-,
   and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —S—, —NH—, —N(C₃₋₆-cycloalkyl)-, —N(C₃₋₆-cycloalkyl-C₁₋₄-alkyl)- or —N(C₁₋₄-alkyl)- and
   where the above-mentioned members may optionally be substituted independently of one another by one or more substituents selected from the group consisting of
   fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl- and C₁₋₆-alkyl-O—,
      and pharmaceutically acceptable salt forms or solvates thereof.

In one embodiment of the invention independent from any other group of the compound of formula (I), D is not oxetanyl, which is bound via the carbon atom next to the oxygen of the oxetanyl, there is no substituent attached to said carbon atom via an integral —CH₂— group.

In a preferred embodiment of the present invention
D is selected from the group D^{1b} consisting of heterocyclyl,
   defined by any of formulas I.1 or I.2 or I.3:

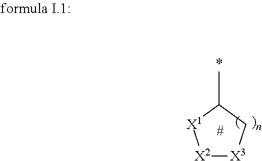

with
n=1, 2, 3;
# meaning that the ring is not aromatic while for n=1, one bond within the ring system optionally may be a double bond and for n=2 or 3 one bond or two bonds within the ring system optionally may be (a) double bond(s), For each occasion the double bond preferably is a C—C double bond. Preferably the ring system is saturated,
X¹, X², X³, independently from each other being CH₂, CHR², CHR³, C(R²)₂, CR²R³, O, NH, NR², or S(O), with r=0, 1, 2, whereby at least one of X¹, X², X³ is O, NH, NR² or S(O)ᵣ, whereby the substituents R² and R³ are selected independently of each other;
The * represents the point of attachment to the nitrogen atom of the pyrazolo ring of formula I;

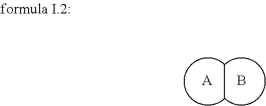

with
A being the ring system of formula I.1;
B being a 3, 4, 5 or 6 membered second ring systems that is anellated to A and that beside the two atoms and one bond it shares with A consists only of carbon atoms and that may be saturated, partially saturated or aromatic, the ring system of formula I.2 may optionally be substituted by one or more substituents selected independently of one another from the group R² and/or optionally substituted by one group R³ and whereby the two ring atoms that are shared by the two ring systems A and B both may be C-atoms, both may be N-atoms or one may be a C- and the other one may be a N-atom. Preferred are two C-atoms, or one C- and one N-atom, and more preferred are two C-atoms. The shared bond may be a single bond or a double bond;

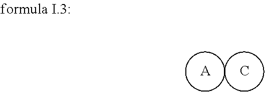

with
A, being the ring system of formula I.1;
C being a 3, 4, 5 or 6 membered second ring systems that is spiro fused to
A and that beside the one atoms it shares with A consists only of carbon atoms and that may be saturated, partially saturated or aromatic whereby the ring system of formula I.3 may optionally be substituted by one or more substituents selected independently of one another from the group R² and/or optionally substituted by one group R³ and whereby the ring atom that is shared by the two ring systems A and C is a C-atom,
   or
D is selected from the group D^{2b} consisting of cyclopropyl, cyclobutyl, cyclopentyl, cyclohexyl, cycloheptyl, cyclopentenyl and cyclohexenyl, wherein the above-mentioned members of the group D^{2b} may optionally be substituted by one or more substituents selected independently of one another from the group R⁴,
   or
D is selected from the group D^{3b} consisting of C₁₋₆-alkyl
   wherein the above-mentioned C₁₋₆-alkyl-group may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
D is selected from the group D^{1c} consisting of heterocyclyl,
   selected from the group of

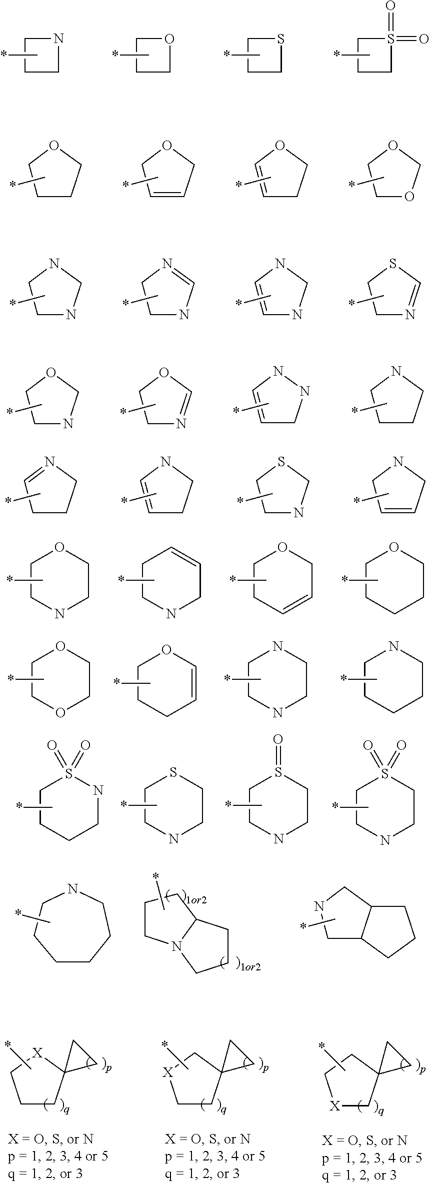

wherein the above-mentioned groups may optionally be substituted by one or more substituents selected independently of one another from the group consisting of C₁₋₃-alkyl.
   or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3c} consisting of C₁₋₅-alkyl
   wherein the above-mentioned C₁₋₅-alkyl-group may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶,

In another preferred embodiment of the present invention
D is selected from the group D^{1d} consisting of heterocyclyl, according to formula I.1 as defined above
   or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3d} consisting of 2-butyl- and 3-pentyl-
   wherein the above-mentioned group D^{3d} may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
D is selected from the group D^{1e} consisting of a monocyclic, non-aromatic, saturated heterocyclic group of 4 to 8, preferably 5, 6 or 7 ring atoms, whereby said ring atoms are carbon atoms and 1, 2 or 3 heteroatom(s), preferably 1 heteroatom, the heteroatom(s) being selected from oxygen, nitrogen and sulphur, the sulphur being in the form of —S(O)ᵣ—with r being 0, 1 or 2, preferably with r being 0 and whereby preferably said heterocyclic group being attached to the scaffold by a carbon ring atom which is not directly attached to said ring heteroatom,
   wherein the above-mentioned groups may optionally be substituted by one or more substituents selected independently of one another from the group consisting of C₁₋₃-alkyl.
      or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3d} consisting of 2-butyl- and 3-pentyl-
   wherein the above-mentioned group D^{3d} may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
D is selected from the group D^{1f} consisting of tetrahydropyranyl, tetrahydrofuranyl, piperidinyl, pyrrolidinyl and piperazinyl, whereby preferably the tetrahydropyranyl is 3- or 4-tetrahydropyranyl, the tetrahydrofuranyl is 3-tetrahydrofuranyl, and the piperidinyl is 3- or 4-piperidinyl,
   wherein the above-mentioned groups may optionally be substituted by one or more substituents selected independently of one another from the group consisting of C₁₋₃-alkyl.
      or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3d} consisting of 2-butyl- and 3-pentyl-
   wherein the above-mentioned group D^{3d} may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
D is selected from the group D^{1g} consisting of tetrahydropyranyl, tetrahydrofuranyl, piperidinyl, and pyrrolindinyl, whereby preferably the tetrahydropyranyl is 3- or 4-tetrahydropyranyl, the tetrahydrofuranyl is 3-tetrahydrofuranyl, and the piperidinyl is 3- or 4-piperidinyl,
   wherein the above-mentioned groups may optionally be substituted by one or more substituents selected independently of one another from the group consisting of C₁₋₃-alkyl.
      or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3d} consisting of 2-butyl- and 3-pentyl-
   wherein the above-mentioned group D^{3d} may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
D is selected from the group D^{1h} consisting of tetrahydropyranyl and tetrahydrofuranyl, preferably 3- or 4-tetrahydropyranyl and 3-tetrahydrofuranyl,
   wherein the above-mentioned groups may optionally be substituted by one or more substituents selected independently of one another from the group consisting of methyl.
      or
D is selected from the group D^{2c} consisting of cyclobutyl, cyclopentyl, and cyclohexyl,
   wherein the above-mentioned members of the group D^{2c} may optionally be substituted by one or more substituents selected independently of one another from the group R⁴,
      or
D is selected from the group D^{3d} consisting of 2-butyl- and 3-pentyl-
   wherein the above-mentioned group D^{3d} may optionally be substituted by one or more substituents selected independently of one another from the group R⁵,
      or
D is selected from the group D^{4b} consisting of phenyl
   wherein the above-mentioned phenyl group may optionally be substituted by one or more substituents selected independently of one another from the group consisting of R⁶,
      or
D is selected from the group D^{5b} consisting of pyridyl
   wherein the above-mentioned pyridyl group may optionally be substituted by one or more substituents selected independently of one another from the group R⁶.

In another preferred embodiment of the present invention
R¹ is selected independently for each R¹ from the group R^{1b} consisting of Hydrogen, fluorine, F₃C—, F₃C—CH₂—, C₁₋₆-alkyl-, C₁₋₃-alkynyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, heterocyclyl-CO—, R¹⁰—O—, R¹⁰—O—C₁₋₃-alkyl-, (R¹⁰)₂N—, R¹⁰O—CO—, (R⁹)₂N—CO—, R¹⁰—CO—(R¹⁰)N—, (R⁹)₂N—CO—(R¹⁰)N—, (R⁹)₂N—CO—O—, and R¹⁰—O—CO—(R¹⁰)N—,
   where the above-mentioned, members F₃C—CH₂—, C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, heterocyclyl-CO—, and R¹⁰—O—C₁₋₃-alkyl-, may optionally be substituted independently of one another by one or more substituents selected independently of one another from the group consisting of fluorine, HO—, F₃C—, HF₂C—, FH₂C—, F₃C—CH₂—, C₁₋₆-alkyl-O—, (R¹⁰)₂N—, (R¹⁰)₂N—C₁₋₃-alkyl-, (R¹⁰)₂N—CO—, C₃₋₆-cycloalkyl-, C₃₋₆-cycloalkyl-C₁₋₄-alkyl- and C₁₋₆-alkyl-.

In another preferred embodiment of the present invention
R¹ is selected independently for each R¹ from the group R^{1c} consisting of C₁₋₆-alkyl-, C₁₋₃-alkynyl-, C₃₋₇-heterocyclyl-C₁₋₆-alkyl-, heteroaryl-, heteroaryl-C₁₋₆-alkyl-, heterocyclyl-CO—, R¹⁰O—CO—, and (R⁹)₂N—CO—,
   where the above-mentioned, members C₁₋₆-alkyl-, C₃₋₇-heterocyclyl-C₁₋₆-alkyl-, heteroaryl-, heterocyclyl-CO—, and heteroaryl-C₁₋₆-alkyl-, may optionally be substituted independently of one another by one or more substituents selected from the group consisting of fluorine, HO—, F₃C—, HF₂C—, FH₂C—, F₃C—CH₂—, C₁₋₆-alkyl-O—, C₁₋₆-alkyl-, (R¹⁰)₂N—, (R¹⁰)₂N—C₁₋₃-alkyl-, C₃₋₆-cycloalkyl-, C₃₋₆-cycloalkyl-C₁₋₄-alkyl- and (R¹⁰)₂N—CO—,

In another preferred embodiment of the present invention
R¹ is selected independently for each R¹ from the group R^{1d} consisting of C₁₋₃-alkyl-, C₁₋₃-alkyl-O—CO—, HO—CO—, (C₁₋₆-alkyl)₂N—CO—, oxadiazolyl, oxazolyl, isoxazolyl, triazolyl, thiazolyl, thienyl, pyrrolyl, furanyl, pyrazolyl, pyridyl, pyridazinyl, pyrimidinyl, oxadiazolyl-C₁₋₃-alkyl-, oxazolyl-C₁₋₃-alkyl-, isoxazolyl-C₁₋₃-alkyl-, triazolyl-C₁₋₃-alkyl-, thiazolyl-C₁₋₃-alkyl-, pyrrolyl-C₁₋₃-alkyl-, furanyl-C₁₋₃-alkyl-, pyrazolyl-C₁₋₃-alkyl-, pyridyl-C₁₋₃-alkyl-, pyridazinyl-C₁₋₃-alkyl-, pyrimidinyl-C₁₋₃-alkyl-, pyrazolyl-C₁₋₃-alkyl-,

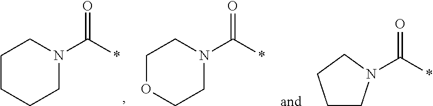

where the above-mentioned members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl-.

In another preferred embodiment of the present invention
R¹ is selected independently for each R^{1e} from the group consisting of C₁₋₃-alkyl-, (C₁₋₆-alkyl)₂N—CO—, oxadiazolyl, pyrazolyl-methyl-,

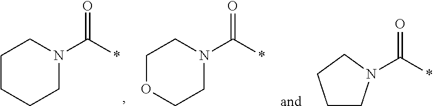

where the above-mentioned, members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl-.

In another preferred embodiment of the present invention
R² is selected from the group R^{2b} consisting of
   H—, fluorine, F₃C—, HF₂C—, FH₂C—, F₃C—CH₂—, C₁₋₆-alkyl-, (R¹⁰)₂N—CO—, R¹⁰—CO—(R¹⁰)N—,
   where the above-mentioned members may optionally be substituted independently of one another by one or more substituents selected from the group consisting of
   fluorine, chlorine, bromine, NC—, O₂N—, F₃C—, HF₂C—, FH₂C—, F₃C—CH₂—, HO—C₁₋₆-alkyl-, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
   and in cases where D is a heterocyclyl group with NR² as ring member, R² shall be
   independently of any other R²: H—, F₃C—CH₂—, HF₂C—CH₂—, C₁₋₆-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl, heteroaryl-C₁₋₆-alkyl-, R¹⁰—O—C₁₋₃-alkyl-, R¹⁰O—CO—, (R¹⁰)₂N—CO—, R¹⁰—CO—, and C₁₋₆-alkyl-SO₂—,
   where the above-mentioned members may optionally be substituted independently of one another by one or more substituents selected from the group consisting of
   fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and) (R¹⁰)₂N—CO—.

In another preferred embodiment of the present invention
R² is selected from the group R^{2c} consisting of
   H—, fluorine, F₃C—, HF₂C—, FH₂C—, F₃C—CH₂—, C₁₋₆-alkyl-, (R¹⁰)₂N—CO—, R¹⁰—CO—(R¹⁰)N—,
   where the above-mentioned members may optionally be substituted independently of one another by one or more substituents selected from the group consisting of
   fluorine, chlorine, bromine, C₁₋₆-alkyl-,
   and in cases where D is a heterocyclyl group with NR² as ring member, R² shall be
   independently of any other R²: H—, F₃C—CH₂—, HF₂C—CH₂—, C₁₋₆-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl, heteroaryl-C₁₋₆-alkyl-, R¹⁰—O—C₁₋₃-alkyl-, R¹⁰O—CO—, (R¹⁰)₂N—CO—, R¹⁰—CO—, and C₁₋₆-alkyl-SO₂—,
   where the above-mentioned members may optionally be substituted independently of one another by one or more substituents selected from the group consisting of
   fluorine, C₁₋₆-alkyl-.

In another preferred embodiment of the present invention
R² is selected from the group R^{2d} consisting of
   H— and C₁₋₆-alkyl-,
   and in cases where D is a heterocyclyl group with NR² as ring member, R² shall be
   independently of any other R²: H—, C₁₋₆-alkyl-, R¹⁰O—CO—, (R¹⁰)₂N—CO—, R¹⁰—CO—, phenyl-CO— and phenyl-O—CO—,
   where the above-mentioned members may optionally be substituted independently of one another by one or more substituents selected from the group consisting of
   fluorine, C₁₋₆-alkyl-.

In another preferred embodiment of the present invention
R² is selected from the group R^{2e} consisting of
   H—, H₃C—,

In another preferred embodiment of the present invention
R³ is selected from the group R^{3b} consisting of
   H—, hydroxy, C₁₋₆-alkyl-O—, whereby C₁₋₆-alkyl-O— may optionally be substituted by one or more fluorine and/or one HO—.

In another preferred embodiment of the present invention
R³ is selected from the group R^{3c} consisting of
   H—.

In another preferred embodiment of the present invention
R⁴ is selected from the group R^{4b} consisting of
   H—, fluorine, NC—, F₃C—, HF₂C—, FH₂C—, F₃C—CH₂—, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N— and R¹⁰—O—CO—(R¹⁰)N—,
   where the above-mentioned members F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl- and (R¹⁰)₂N—C₁₋₃-alkyl-, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—.

In another preferred embodiment of the present invention
R⁴ is selected from the group R^{4c} consisting of
   H—, fluorine, F₃C—, C₁₋₆-alkyl-, aryl, HO—, C₁₋₆-alkyl-O—, C₁₋₆-alkyl-O—C₂₋₃-alkyl-, (R¹⁰)₂N—, (R¹⁰)₂N—C₁₋₃-alkyl-, (R¹⁰)₂N—CO—, R¹⁰—CO—(R¹⁰)N—, (R¹⁰)₂N—CO—(R¹⁰)N— and R¹⁰—O—CO—(R¹⁰)N—,
   where the above-mentioned members C₁₋₆-alkyl-, aryl, C₁₋₆-alkyl-O—, C₁₋₆-alkyl-O—C₂₋₃-alkyl- and (R¹⁰)₂N—C₁₋₃-alkyl- may optionally be substituted independently of one another by one or more substituents selected from the group consisting of fluorine, chlorine, bromine, NC—, C₁₋₃-alkyl-, and F₃C—.

In another preferred embodiment of the present invention
R⁴ is selected from the group R^{4d} consisting of
   H—, fluorine, methyl-, HO—, CH₃—O—, phenyl-, H₂N—, C₁₋₆-alkyl-O—CO—(H)N—, C₁₋₆-alkyl-CO—(H)N—, phenyl-CO—(H)N—,
   where the above-mentioned members methyl-, CH₃—O—, phenyl-, H₂N—, C₁₋₆-alkyl-O—CO—(H)N—, C₁₋₆-alkyl-CO—(H)N—, phenyl-CO—(H)N— may optionally be substituted independently of one another by one or more fluorine.

In another preferred embodiment of the present invention

R⁴ is selected from the group R^{4e} consisting of
H—, and fluorine.

In another preferred embodiment of the present invention
R⁵ is selected from the group R^{5b} consisting of
   H—, fluorine, NC—, F₃C—, HF₂C—, FH₂C—, F₃C—CH₂—, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, and R¹⁰—O—CO—(R¹⁰)N—,
   where the above-mentioned, members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl-, and (R¹⁰)₂N—C₁₋₃-alkyl- may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,

In another preferred embodiment of the present invention
R⁵ is selected from the group R^{5c} consisting of
   H—, fluorine, F₃C—, C₁₋₆-alkyl aryl, R¹⁰—O—, R¹⁰—O—C₁₋₃-alkyl-, (R¹⁰)₂N—, (R¹⁰)₂N—C₁₋₃-alkyl-, (R¹⁰)₂N—CO—, R¹⁰—CO—(R¹⁰)N—, (R¹⁰)₂N—CO—(R¹⁰)N—, and R¹⁰—O—CO—(R¹⁰)N—,
   where the above-mentioned, members C₁₋₆-alkyl-, aryl R¹⁰—O—C₁₋₃-alkyl-, and (R¹⁰)₂N—C₁₋₃-alkyl- may optionally be substituted independently of one another by one or more substituents selected from the group consisting of fluorine, chlorine, bromine, NC—, F₃C—, and C₁₋₆-alkyl-,

In another preferred embodiment of the present invention
R⁵ is selected from the group R^{5d} consisting of H— and fluorine,

In another preferred embodiment of the present invention
R⁶ is selected from the group R^{6b} consisting of
   H—, fluorine, chlorine, bromine, HO—, NC—, F₃C—, HF₂C—, FH₂C—, F₃C—CH₂—, carboxy-, C₁₋₆-alkyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)²N—, (R¹⁰)₂N—C₁₋₃-alkyl-, R¹⁰O—CO—, (R¹⁶)₂N—CO—, R¹⁰—CO—(R¹⁰)N—, R¹⁰—CO—, (R¹⁶)₂N—CO—(R¹⁰)N—, and R¹⁰—O—CO—(R¹⁰)N—,
   where the above-mentioned, members HF₂C—, FH₂C—, F₃C—CH₂—, C₁₋₆-alkyl-, C₁₋₆-alkyl-S—C₁₋₃-alkyl-, C₃₋₇-cycloalkyl-, C₃₋₇-cycloalkyl-C₁₋₆-alkyl C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl- and (R¹⁰)₂N—C₁₋₃-alkyl-, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,

In another preferred embodiment of the present invention
R⁶ is selected from the group R^{6c} consisting of
   H—, fluorine, chlorine, bromine, HO—, NC—, F₃C—, C₁₋₆-alkyl-, C₁₋₆-alkyl-S—, C₁₋₆-alkyl-S—C₁₋₃-alkyl-, C₃₋₇-cycloalkyl-, C₃₋₇-heterocyclyl-, aryl heteroaryl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO— and R¹⁰—CO—(R¹⁰)N—,
   where the above-mentioned, members C₁₋₆-alkyl-, C₁₋₆-alkyl-S—C₁₋₃-alkyl-, C₃₋₇-cycloalkyl-, C₃₋₇-heterocyclyl-, aryl, heteroaryl-, R¹⁰—O—C₁₋₃-alkyl- and (R¹⁰)₂N—C₁₋₃-alkyl-, may optionally be substituted independently of one another by one or more substituents selected from the group consisting of fluorine, chlorine, bromine, NC—, F₃C—, and C₁₋₆-alkyl-,

In another preferred embodiment of the present invention
R⁶ is selected from the group R^{6d} consisting of
   H—, fluorine, chlorine, bromine, F₃C—, C₁₋₆-alkyl-, and R¹⁰—O—,
   where the above-mentioned, member C₁₋₆-alkyl-, may optionally be substituted independently of one another by one or more substituents selected from the group consisting of fluorine,

In another preferred embodiment of the present invention
R⁶ is selected from the group R^{6e} consisting of
   H—, fluorine, chlorine, bromine, F₃C—, H₃O—, and H₃C—O—,
   where the above-mentioned, member H₃O—, may optionally be substituted independently of one another by one or more substituents selected from the group consisting of fluorine,

In another preferred embodiment of the present invention
R⁹ is selected independently for each R⁹ from the group R^{9b} consisting of H—, C₁₋₆-alkyl-, C₂₋₆-alkinyl-, C₃₋₇-cycloalkyl-, C₃₋₇-cycloalkyl-C₁₋₃-alkyl-, aryl, aryl-C₁₋₃-alkyl-, heteroaryl, and heteroaryl-C₁₋₃-alkyl-,
   where the above-mentioned members C₁₋₆-alkyl-, C₂₋₆-alkinyl-, C₃₋₇-cycloalkyl-, C₃₋₇-cycloalkyl-C₁₋₃-alkyl-, aryl, aryl-C₁₋₃-alkyl-, heteroaryl, and heteroaryl-C₁₋₃-alkyl- may optionally be substituted independently of one another by one or more substituents selected independently from one another from the group consisting of
   fluorine, NC—, F₃C—, HF₂C—, FH₂C—, F₃C—CH₂—, CH₃—O—C₁₋₆-alkyl-, C₁₋₆-alkyl-O— and C₁₋₆-alkyl-.

In another preferred embodiment of the present invention
R⁹ is selected independently for each R⁹ from the group R^{9c} consisting of H—, C₁₋₆-alkyl-, C₂₋₆-alkinyl-, C₃₋₇-cycloalkyl-, aryl and heteroaryl, and
   where the above-mentioned members C₁₋₆-alkyl-, C₂₋₆-alkinyl-, C₃₋₇-cycloalkyl-, aryl and heteroaryl may optionally be substituted independently of one another by one or more substituents selected independently of one another from the group consisting of fluorine, NC—, F₃C—, HF₂C—, FH₂C—, F₃C—CH₂—, CH₃—O—C₁₋₆-alkyl-, and C₁₋₆-alkyl-O— and C₁₋₆-alkyl-.

In another preferred embodiment of the present invention
R⁹ is selected independently for each R⁹ from the group R^{9d} consisting of H—, C₁₋₆-alkyl-, phenyl, and pyridyl, and
   where the above-mentioned members C₁₋₆-alkyl-, phenyl, and pyridyl may optionally be substituted independently of one another by one or more substituents selected independently of one another from the group consisting of
   fluorine, F₃C—, HF₂C—, FH₂C—, F₃C—CH₂—, CH₃—O—C₁₋₆-alkyl-, C₁₋₆-alkyl-O— and C₁₋₆-alkyl-.

In another preferred embodiment of the present invention
R⁹ is selected independently for each R⁹ from the group R^{9e} consisting of H—, methyl-, ethyl- and tert.-butyl, and
   where the above-mentioned members methyl-, ethyl- and tert.-butyl may optionally be substituted independently of one another by one or more substituents selected from the group consisting of fluorine.

In another preferred embodiment of the present invention
R¹⁰ is selected independently for each R¹⁰ from the group R^{10b} consisting of H— (but not in case is part of a group being selected from R¹⁰O—CO—, R¹⁰—SO₂— or R¹⁰—CO—), C₁₋₆-alkyl-, C₃₋₇-cycloalkyl-, C₃₋₇-cycloalkyl-C₁₋₃-alkyl-, aryl and heteroaryl,
   and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —NH—, —N(C₃₋₆-cycloalkyl)-, —N(C₃₋₆-cycloalkyl-C₁₋₄-alkyl)- or —N(C₁₋₄-alkyl)- and
   where the above-mentioned members may optionally be substituted independently of one another by one or more substituents selected from the group consisting of
   fluorine, NC—, F₃C—, HF₂C—, FH₂C—, F₃C—CH₂—, CH₃—O—C₁₋₆-alkyl-, C₁₋₆-alkyl-, and C₁₋₆-alkyl-O—.

In another preferred embodiment of the present invention
R¹⁰ is selected independently for each R¹⁰ from the group R^{10C} consisting of H— (but not in case is part of a group being selected from R¹⁰O—CO—, R¹⁰—SO₂— or R¹⁰—CO—), C₁₋₆-alkyl-, C₃₋₇-cycloalkyl-, aryl and heteroaryl,
   and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —NH—, —N(C₃₋₆-cycloalkyl)-, —N(C₃₋₆-cycloalkyl-C₁₋₄-alkyl)- or —N(C₁₋₄-alkyl)- and
   where the above-mentioned members may optionally be substituted independently of one another by one or more substituents selected from the group consisting of
   fluorine, NC—, F₃C—, HF₂C—, FH₂C—, F₃C—CH₂—, CH₃—O—C₁₋₆-alkyl-, C₁₋₆-alkyl-, and C₁₋₆-alkyl-O—.

In another preferred embodiment of the present invention
R¹⁰ is selected independently for each R¹⁰ from the group R^{10d} consisting of H— (but not in case is part of a group being selected from R¹⁰O—CO—, R¹⁰—SO₂— or R¹⁰—CO—), C₁₋₆-alkyl-, phenyl, and pyridyl,
   where the above-mentioned members may optionally be substituted independently of one another by one or more substituents selected from the group consisting of
   fluorine, F₃C—, HF₂C—, FH₂C—, F₃C—CH₂—, CH₃—O—C₁₋₆-alkyl-, C₁₋₆-alkyl-, and C₁₋₆-alkyl-O—.

In another preferred embodiment of the present invention
R¹⁰ is selected independently for each R¹⁰ from the group R^{10e} consisting of H— (but not in case is part of a group being selected from R¹⁰O—CO—, R¹⁰—SO₂— or R¹⁰—CO—), methyl-, ethyl- and tert.-butyl,
   where the above-mentioned members may optionally be substituted independently of one another by one or more substituents selected from the group consisting of fluorine.

Any and each of the above definitions for x, y, L, D and R¹ to R¹⁰ may be combined with each other.

In another preferred embodiment of the present invention
D is selected from the group consisting of cyclopentyl, cyclohexyl, 3-pentyl-, phenyl, tetrahydropyranyl, tetrahydrofuranyl and pyridyl, wherein the above-mentioned cyclopentyl, cyclohexyl, 3-pentyl-, phenyl, and pyridyl groups may optionally be substituted by one or more substituents selected independently of one another from the group consisting of fluorine, chlorine or methyl and
   wherein the above-mentioned tetrahydropyranyl and tetrahydrofuranyl groups may optionally be substituted by one or more substituents selected independently of one another from the group consisting of methyl.
L is selected from the integers 0, 1 and 2,
x is selected from the integers 0, 1, 2 and 3,
y is 0,
   and
R¹ is selected independently for each R¹ from the group R^{1d} consisting of C₁₋₃-alkyl-, C₁₋₃-alkyl-O—CO—, HO—CO—, (C₁₋₆-alkyl)₂N—CO—, oxadiazolyl, oxazolyl, isoxazolyl, triazolyl, thiazolyl, thienyl, pyrrolyl, furanyl, pyrazolyl, pyridyl, pyridazinyl, pyrimidinyl, oxadiazolyl-C₁₋₃-alkyl-, oxazolyl-C₁₋₃-alkyl-, isoxazolyl-C₁₋₃-alkyl-, triazolyl-C₁₋₃-alkyl-, thiazolyl-C₁₋₃-alkyl-, pyrrolyl-C₁₋₃-alkyl-, furanyl-C₁₋₃-alkyl-, pyrazolyl-C₁₋₃-alkyl-, pyridyl-C₁₋₃-alkyl-, pyridazinyl-C₁₋₃-alkyl-, pyrimidinyl-C₁₋₃-alkyl-, pyrazolyl-C₁₋₃-alkyl-,

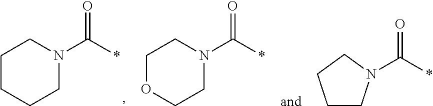

where the above-mentioned, members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl-.

In another preferred embodiment of the present invention
D is selected from the group consisting of cyclopentyl, cyclohexyl, 3-pentyl-, phenyl and pyridyl,
   wherein the above-mentioned groups may optionally be substituted by one or more substituents selected independently of one another from the group consisting of fluorine, chlorine or methyl.
L is 1,
x is selected from the integers 1 and 2,
y is 0,
   and
R¹ is selected independently for each R^{1e} from the group consisting of C₁₋₃-alkyl-, (C₁₋₆-alkyl)₂N—CO—, oxadiazolyl, pyrazolyl-methyl-,

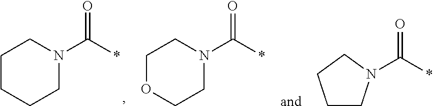

where the above-mentioned, members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl-.

In another preferred embodiment of the invention in the two directly above mentioned embodiments
D is selected from the group consisting of cyclopentyl, cyclohexyl and pyridyl, wherein the above-mentioned groups may optionally be substituted by one or more substituents selected independently of one another from the group consisting of fluorine, chlorine or methyl.

The following matrix I shows further embodiments of the inventions that are considered preferred. L, x and y are as defined below.

### Matrix I:

| | | | | |
|---|---|---|---|---|
| D^{1b/2b/3b/4b/5b}/R^{1b}/ | D^{1c/2c/3c/4b/5b}/R^{1c}/ | | | |
| R^{2b}/R^{3b}/R^{4b}/R^{5b}/ | R^{4c}/R^{5c}/R^{6c}/R^{9c}/ | | | |
| R^{6b}/R^{9b}/R^{10b} | R^{10c} | | | |
| D^{1d/2c/3d/4b/5b}/R^{1d}/ | D^{1e/2c/3d/4b/5b}/R^{1e}/ | D^{1f/2c/3d/4b/5b}/R^{1e}/ | D^{1g/2c/3d/4b/5b}/R^{1e}/ | D^{1h/2c/3d/4b/5b}/R^{1e}/ |
| R^{2d}/R^{3c}/R^{4d}/R^{5d}/ | R^{4e}/R^{5d}/R^{6e}/R^{9e}/ | R^{4e}/R^{5d}/R^{6e}/R^{9e}/ | R^{4e}/R^{5d}/R^{6e}/R^{9e}/ | R^{4e}/R^{5d}/R^{6e}/R^{9e}/ |
| R^{6d}/R^{9d}/R^{10d} | R^{10e} | R^{10e} | R^{10e} | R^{10e} |
| D^{1h/2c/3d/4b/5b}/R^{1a}/ | D^{1h/2c/3d/4b/5b}/R^{1b}/ | D^{1h/2c/3d/4b/5b}/R^{1c}/ | D^{1h/2c/3d/4b/5b}/ | D^{1h/2c/3d/4b/5b}/R^{1e}/ |
| R^{4a}/R^{5a}/R^{6a}/R^{9a}/ | R^{4b}/R^{5b}/R^{6b}/R^{9b}/ | R^{4c}/R^{5c}/R^{6c}/R^{9c}/ | R^{1d}/R^{4d}/R^{5d}/R^{6d}/ | R^{4e}/R^{5d}/R^{6e}/R^{9e}/ |
| R^{10a} | R^{10b} | R^{10c} | R^{9d}/R^{10d} | R^{10e} |
| D^{1a}/R^{1a}/R^{2a}/ | D^{1a}/R^{1b}/R^{2b}/ | D^{1a}/R^{1c}/R^{2c}/ | D^{1a}/R^{1d}/R^{2d}/R^{3c} | D^{1a}/R^{1e}/R^{2e}/R^{3c} |
| R^{3a}/R^{9a}/R^{10a} | R^{3b}/R^{9b}/R^{10b} | R^{3c}/R^{9c}/R^{10c} | | |
| D^{1b}/R^{1a}/R^{2a}/ | D^{1b}/R^{1b}/R^{2b}/ | D^{1b}/R^{1c}/R^{2c}/ | D^{1b}/R^{1d}/R^{2d}/R^{3c} | D^{1b}/R^{1e}/R^{2e}/R^{3c} |
| R^{3a}/R^{9a}/R^{10a} | R^{3b}/R^{9b}/R^{10b} | R^{3c}/R^{9c}/R^{10c} | | |
| D^{1c}/R^{1a}/R^{9a}/R^{10a} | D^{1c}/R^{1b}/R^{9b}/R^{10b} | D^{1c}/R^{1c}/R^{9c}/R^{10c} | D^{1c}/R^{1d} | D^{1c}/R^{1e} |
| D^{1d}/R^{1a}/R^{2a}/ | D^{1d}/R^{1b}/R^{2b}/ | D^{1d}/R^{1c}/R^{2c}/ | D^{1d}/R^{1d}/R^{2d}/R^{3c} | D^{1d}/R^{1e}/R^{2e}/R^{3c} |
| R^{3a}/R^{9a}/R^{10a} | R^{3b}/R^{9b}/R^{10b} | R^{3c}/R^{9c}/R^{10c} | | |
| D^{1e}/R^{1a}/R^{9a}/R^{10a} | D^{1e}/R^{1b}/R^{9b}/R^{10b} | D^{1e}/R^{1c}/R^{9c}/R^{10c} | D^{1e}/R^{1d} | D^{1e}/R^{1e} |
| D^{1f}/R^{1a}/R^{9a}/R^{10a} | D^{1f}/R^{1b}/R^{9b}/R^{10b} | D^{1f}/R^{1c}/R^{9c}/R^{10c} | D^{1f}/R^{1d} | D^{1f}/R^{1e} |
| D^{1g}/R^{1a}/R^{9a}/R^{10a} | D^{1g}/R^{1b}/R^{9b}/R^{10b} | D^{1g}/R^{1c}/R^{9c}/R^{10c} | D^{1g}/R^{1d} | D^{1g}/R^{1e} |
| D^{1h}/R^{1a}/R^{9a}/R^{10a} | D^{1h}/R^{1b}/R^{9b}/R^{10b} | D^{1h}/R^{1c}/R^{9c}/R^{10c} | D^{1h}/R^{1d} | D^{1h}/R^{1e} |

and the salts thereof, preferably pharmaceutically acceptable salts thereof, solvates thereof or the solvates of the aforementioned salts thereof.

Within the meaning of the present invention the term “D^{1b/2b/3b/4b/5b}/R^{1b}/R^{2b}/R^{3b}/R^{4b}/R^{5b}/R^{6b}/R^{9b}/R^{10b}” as given in above table embraces all such compounds of formula (I) wherein the substituent represented in formula (I) by “D” is selected from one of the groups defined by D^{1b}, D^{2b}, D^{3b}, D^{4b} and D^{5b}, the substituent represented by R¹ is selected from one of the groups defined by R^{1b}, the substituent represented by R² is selected from one of the groups defined by R^{2b}, the substituent represented by R⁴ is selected from one of the groups defined by R^{4b}, the substituent represented by R⁵ is selected from one of the groups defined by R^{5b}, the substituent represented by R⁶ is selected from one of the groups defined by R^{6b}, the substituent represented by R⁹ is selected from one of the groups defined by R^{9b}, and the substituent represented by R¹⁰ is selected from one of the groups defined by R^{10b}.

For all embodiments L is selected from the integers 0, 1, 2 and 3, preferably 1 and 2; x is selected from the integers 0, 1, 2, 3 and 4, preferably 0, 1, 2 and 3, more preferably 1 and 2; y is selected from the integers 0, 1 and 2, preferably 0 and 1.

In another preferred embodiment of the present invention related to the compounds of formulae I through 47 as listed below in table 1

TABLE 1

| No. | Compound |
|---|---|
| 1 | 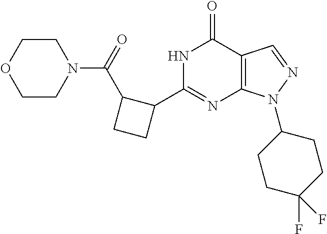 |
| 2 | 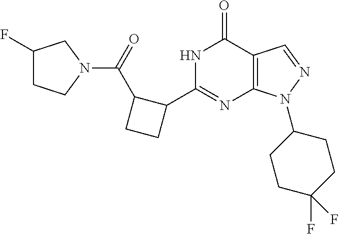 |
| 3 | 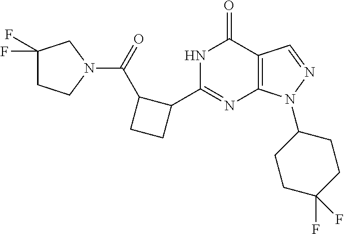 |
| 4 | 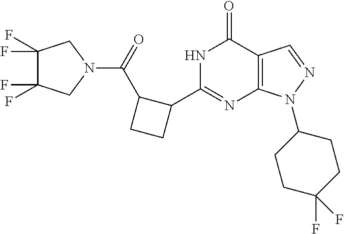 |
| 5 | 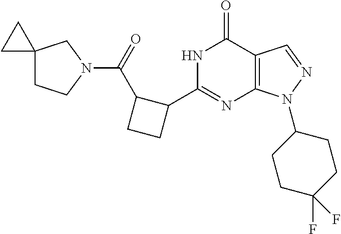 |
| 6 | 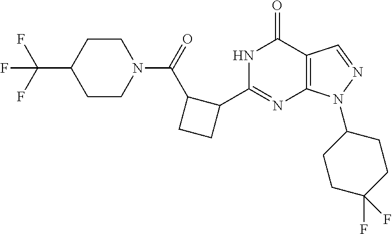 |
| 7 | 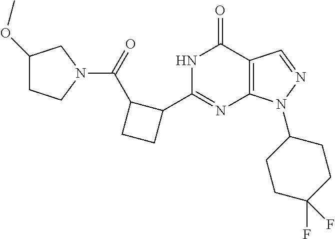 |
| 8 | 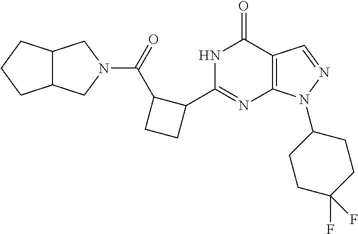 |
| 9 | 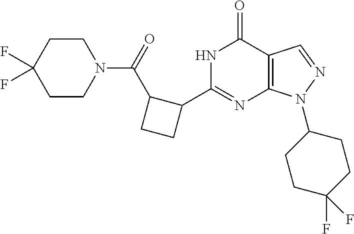 |
| 10 | 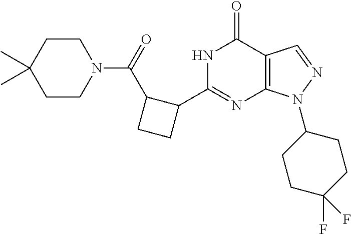 |
| 11 | 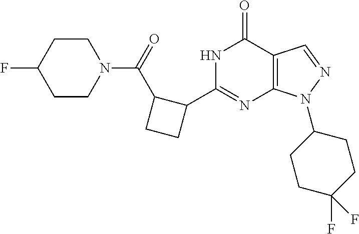 |
| 12 | 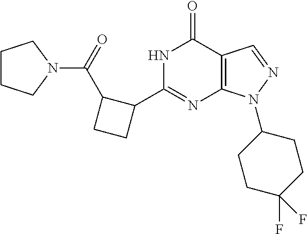 |
| 13 | 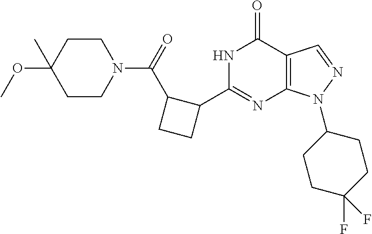 |
| 14 | 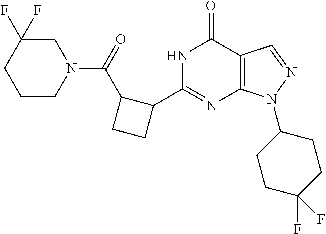 |
| 15 | 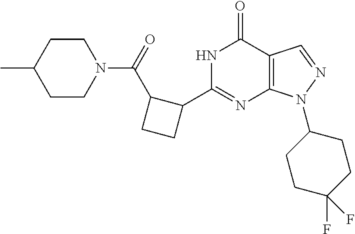 |
| 16 | 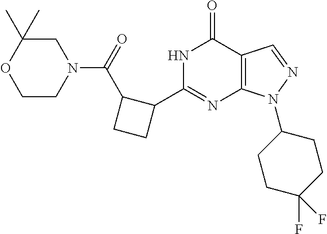 |
| 17 | 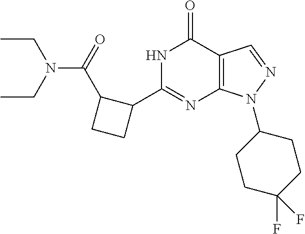 |
| 18 | 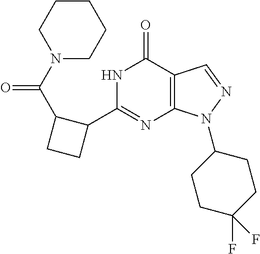 |
| 19 | 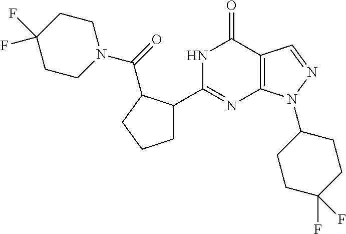 |
| 20 | 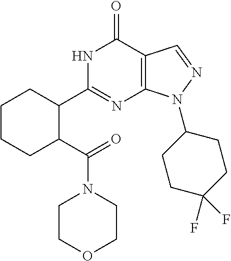 |
| 21 | 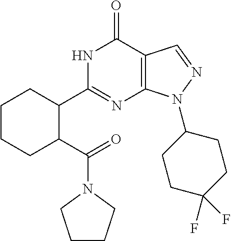 |
| 22 | 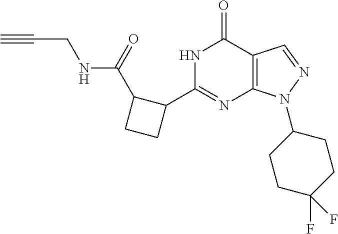 |
| 23 | 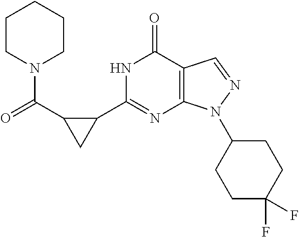 |
| 24 | 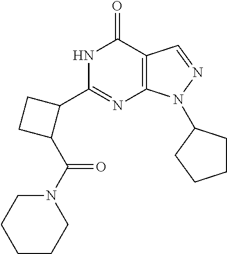 |
| 25 | 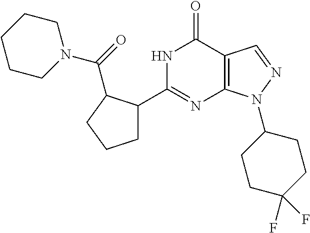 |
| 26 | 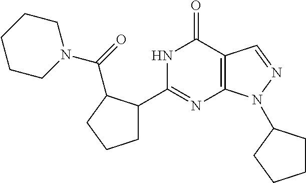 |
| 27 | 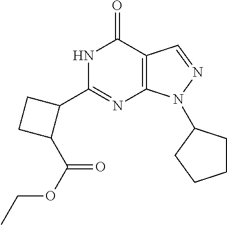 |
| 28 | 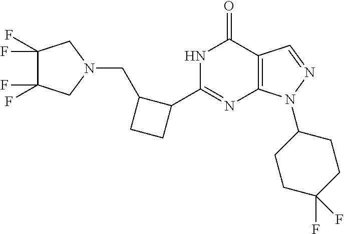 |
| 29 | 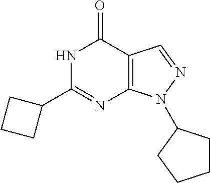 |
| 30 | 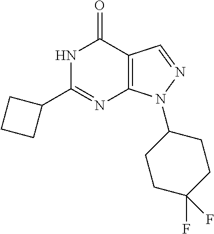 |
| 31 | 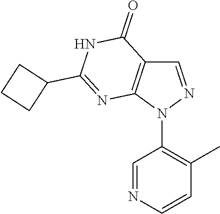 |
| 32 | 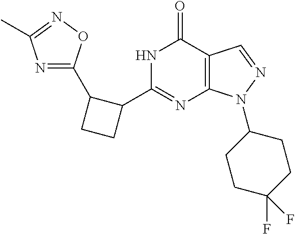 |
| 33 | 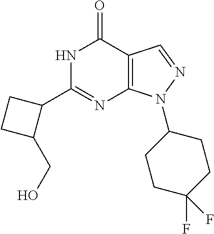 |
| 34 | 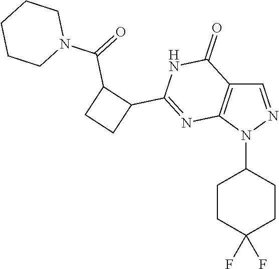 |
| 35 | 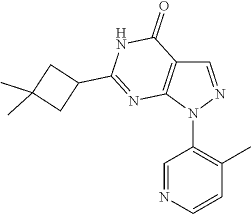 |
| 36 | 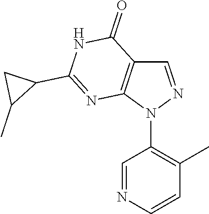 |
| 37 | 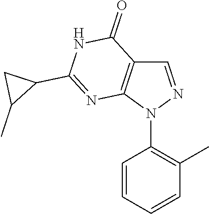 |
| 38 | 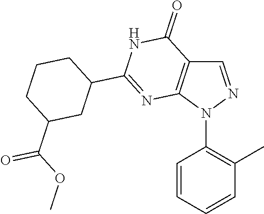 |
| 39 | 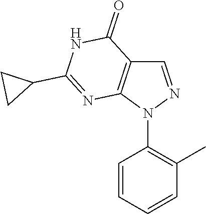 |
| 40 | 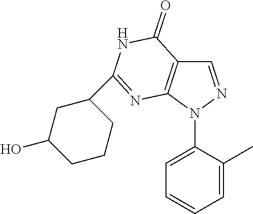 |
| 41 | 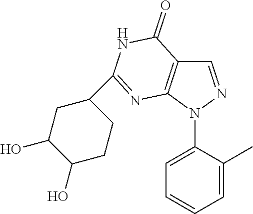 |
| 42 | 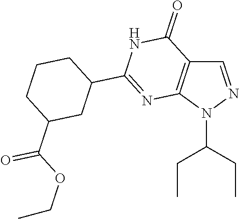 |
| 43 | 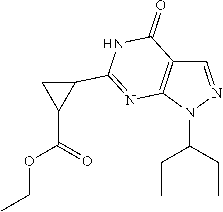 |
| 44 | 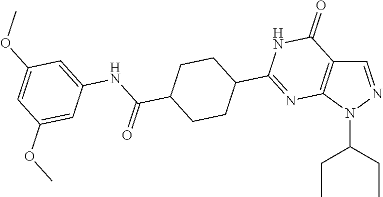 |
| 45 | 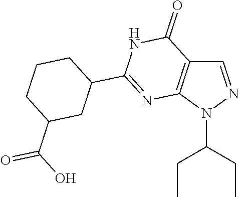 |
| 46 | 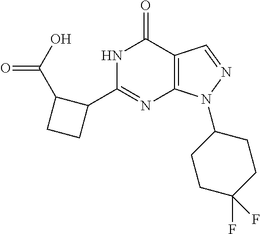 |
| 47 | 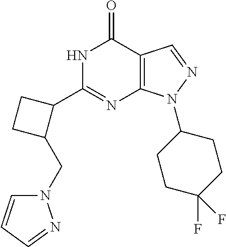 |

and the salts thereof, preferably pharmaceutically acceptable salts thereof, solvates thereof or the solvates of the aforementioned salts thereof.

### USED TERMS AND DEFINITIONS

Terms not specifically defined herein should be given the meanings that would be given to them by a person skilled in the art in light of the disclosure and the context. Examples include that specific substituents or atoms are presented with their 1 or 2 letter code, like H for hydrogen, N for nitrogen, C for carbon, O for oxygen, S for sulphur and the like. Optionally but not mandatorily the letter is followed by a hyphen to indicate a bond. As used in the specification, unless specified to the contrary, the following terms have the meaning indicated and the following conventions are adhered to.

In the groups, radicals, or moieties defined below, the number of carbon atoms is often specified preceding the group, for example, C₁₋₆ alkyl means an alkyl group or alkyl radical having 1 to 6 carbon atoms. In general, for groups comprising two or more subgroups, the last named group is the radical attachment point, for example, “alkyl-O—” means a monovalent radical of the formula alkyl-O—, which is attached via the oxygen atom (alkoxy). If the term of a substituent starts or ends with a minus sign or hyphen, i.e.-, this sign emphasises the attachment point like in the aforementioned example alkyl-O—, where the “O” is linked to the group of which the alkyl-O— is a substituent. Unless otherwise specified below, conventional definitions of terms control and conventional stable atom valences are presumed and achieved in all formulas and groups.

In general, all “tautomeric forms and isomeric forms and mixtures”, whether individual geometric isomers or optical isomers or racemic or non-racemic mixtures of isomers, of a chemical structure or compound are intended, unless the specific stereochemistry or isomeric form is specifically indicated in the compound name or structure.

The term “substituted” as used herein explicitly or implicitly, means that any one or more hydrogen(s) on the designated atom is replaced with a member of the indicated group of substituents, provided that the designated atom's normal valence is not exceeded. In case a substituent is bound via a double bond, e.g. an oxo substituent, such substituent replaces two hydrogen atoms on the designated atom. The substitution shall result in a stable compound. “Stable” in this context preferably means a compound that from a pharmaceutical point of view is chemically and physically sufficiently stable in order to be used as an active pharmaceutical ingredient of a pharmaceutical composition.

If a substituent is not defined, it shall be hydrogen.

By the term “optionally substituted” is meant that either the corresponding group is substituted or it is not. Accordingly, in each occasion where this term is used, the non-substituted variation is a more pronounced aspect of the invention, i.e. preferably there are no such optional substituents.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salt(s)” refer to derivatives of the disclosed compounds wherein the parent compound is modified by making acid or base salts thereof. Preferably addition salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid, hydrobromic acid, sulfuric acid, sulfamic acid, phosphoric acid, nitric acid, and the like; and the salts prepared from organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, and the like. As the compounds of the present invention may have both, acid as well as basic groups, those compounds may therefore be present as internal salts too.

The pharmaceutically acceptable salts of the present invention can be synthesized from the parent compound which contains a basic or acidic moiety by conventional chemical methods. Generally, such salts can be prepared by reacting the free acid or base form of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred.

“Prodrugs” are considered compounds that release an active parent drug of the present invention in vivo when such prodrug is administered to a mammalian subject. Prodrugs according to the present invention are prepared by modifying functional groups present in the compound in such a way that these modifications are retransformed to the original functional groups under physiological conditions. Prodrugs include compounds of the present invention wherein a hydroxy, amino, or sulfhydryl group is bound to any group that, when the prodrug of the present invention is administered to a mammalian subject, is retransformed to free said hydroxyl, amino, or sulfhydryl group. Examples of prodrugs include, but are not limited to, acetate, formate and benzoate derivatives of alcohol and amine functional groups in the compounds of the present invention.

“Metabolites” are considered as derivatives of the compounds according to the present invention that are formed in vivo. Active metabolites are such metabolites that cause a pharmacological effect. It will be appreciated that metabolites of the compounds according to the present inventions are subject to the present invention as well, in particular active metabolites.

Some of the compounds may form “solvates”. For the purposes of the invention the term “solvates” refers to those forms of the compounds which form, in the solid or liquid state, a complex by coordination with solvent molecules. Hydrates are a specific form of solvates in which the coordination takes place with water. According to the present invention, the term preferably is used for solid solvates, such as amorphous or more preferably crystalline solvates.

“Scaffold”: The scaffold of the compounds according to the present invention is represented by the following core structure, the numeration of the ring members thereof is indicated in bold:

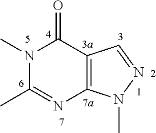

It will be evident for the skilled person in the art, that this scaffold can be described by its tautomeric “enol” form

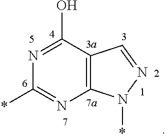

In the context of the present invention both structural representations of the scaffold shall be considered the subject of the present invention, even if only one of the two representatives is presented. It is believed that for the majority of compounds under ambient conditions and therewith under conditions which are the relevant conditions for a pharmaceutical composition comprising said compounds, the equilibrium of the tautomeric forms lies on the side of the pyrazolopyrimdin-4-one representation. Therefore, all embodiments are presented as pyrazolopyrimdin-4-one-derivatives or more precisely as pyrazolo[3,4-d]pyrimidin-4-one derivatives.

“Bonds”: If within a chemical formula of a ring system or a defined group a substituent is directly linked to an atom or a group like “RyR” in below formula this shall mean that the substituent is only attached to the corresponding atom. If however from another substituent like “RxR” a bond is not specifically linked to an atom of the ring system but drawn towards the centre of the ring or group this means that this substituent “RxR” may be linked to any meaningful atom of the ring system/group unless stated otherwise.

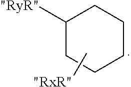

The bond symbol “—” (=minus sign) or the symbol “—*” (=minus sign followed by an asterisk sign) stands for the bond through which a substituent is bound to the corresponding remaining part of the molecule/scaffold. In cases in that minus sign does not seem to be sufficiently clear, an asterisk is added to the bond symbol “—” in order to determine the point of attachment of said bond with the corresponding main part of the molecule/scaffold.

In general, the bond to one of the herein defined heterocyclyl or heteroaryl groups may be effected via a C atom or optionally an N atom.

The term “aryl” used in this application denotes a phenyl, biphenyl, indanyl, indenyl, 1,2,3,4-tetrahydronaphthyl or naphthyl group, preferably it denotes a phenyl or naphtyl group, more preferably a phenyl group. This definition applies for the use of “aryl” in any context within the present description in the absence of a further definition.

The term “C₁₋ₙ-alkyl” denotes a saturated, branched or unbranched hydrocarbon group with 1 to n C atoms, wherein n is a figure selected from the group of 2, 3, 4, 5, 6, 7, 8, 9, or 10, preferably from the group of 2, 3, 4, 5, or 6, more preferably from the group of 2, 3, or 4. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl etc. As will be evident from the context, such C₁₋ₙ-alkyl group optionally can be substituted.

This definition applies for the use of “alkyl” in any reasonable context within the present description in the absence of a further definition.

In cases in which the term “C₁₋ₙ-alkyl” is used in the middle of two other groups/substituents, like for example in “C₁₋ₙcycloalkyl-C₁₋ₙ-alkyl-O—”, this means that the “C₁₋ₙ-alkyl”-moiety bridges said two other groups. In the present example it bridges the C₁₋ₙcycloalkyl with the oxygen like in “cyclopropyl-methyl-oxy-”. It will be evident, that in such cases “C₁₋ₙ-alkyl” has the meaning of a “C₁₋ₙ-alkylene” spacer like methylene, ethylene etc. The groups that are bridged by “C₁₋ₙ-alkyl” may be bound to “C₁₋ₙ-alkyl” at any position thereof. Preferably the right hand group is located at the distal right hand end of the alkyl group and left hand group at the distal left hand side of the alkyl group. The same applies for other substituents.

The term “C₂₋ₙ-alkenyl” denotes a branched or unbranched hydrocarbon group with 2 to n C atoms and at least one C═C group (i.e. carbon-carbon double bond), wherein n preferably has a value selected from the group of 3, 4, 5, 6, 7, or 8, more preferably 3, 4, 5, or 6, more preferably 3 or 4. Examples of such groups include ethenyl, 1-propenyl, 2-propenyl, iso-propenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl etc. As will be evident from the context, such C₂₋ₙ-alkenyl group optionally can be substituted.

This definition applies for the use of “alkenyl” in any reasonable context within the present description in the absence of a further definition.

In cases in which the term “C₂₋ₙ-alkenyl” is used in the middle of two other groups/substituents, the analogue definition as for C₁₋ₙ-alkyl applies.

The term “C₂₋ₙ-alkynyl” denotes a branched or unbranched hydrocarbon group with 2 to n C atoms and at least one C≡C group (i.e. a carbon-carbon triple bond), wherein n preferably has a value selected from the group of 3, 4, 5, 6, 7, or 8, more preferably 3, 4, 5, or 6, more preferably 3 or 4. Examples of such groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl etc. As will be evident from the context, such C₂₋ₙ-alkynyl group optionally can be substituted.

This definition applies for the use “alkynyl” in any reasonable context within the present description in the absence of a further definition.

In cases in which the term “C₂₋ₙ-alkynyl” is used in the middle of two other groups/substituents, the analogue definition as for C₁₋ₙ-alkyl applies.

The term “C₃₋ₙ-cycloalkyl” denotes a saturated monocyclic group with 3 to n C ring atoms. n preferably has a value of 4 to 8 (=4, 5, 6, 7, or 8), more preferably 4 to 7, more preferably such C₃₋ₙ-cycloalkyl is 5 or 6 membered. Examples of such groups include cyclopropyl, cyclobutyl, cyclopentyl, cyclohexyl, cycloheptyl etc. This definition applies for “cycloalkyl” in any reasonable context within the present description in the absence of a further definition.

The term “halogen” denotes an atom selected from among F, Cl, Br, and I.

The term “heteroaryl” used in this application denotes a heterocyclic, mono- or bicyclic aromatic ring system which includes within the ring system itself in addition to at least one C atom one or more heteroatom(s) independently selected from N, O, and/or S. A monocyclic ring system preferably consists of 5 to 6 ring members, a bicyclic ring system preferably consists of 8 to 10 ring members. Preferred are heteroaryls with up to 3 heteroatoms, more preferred up to 2 heteroatoms, more preferred with 1 heteroatom. Preferred heteroatom is N. Examples of such moieties are benzimidazolyl, benzisoxazolyl, benzo[1,4]-oxazinyl, benzoxazol-2-onyl, benzofuranyl, benzoisothiazolyl, 1,3-benzodioxolyl, benzothiadiazolyl, benzothiazolyl, benzothienyl, benzoxadiazolyl, benzoxazolyl, chromanyl, chromenyl, chromonyl, cinnolinyl, 2,3-dihydrobenzo[1,4]dioxinyl, 2,3-dihydrobenzofuranyl, 3,4-dihydrobenzo[1,4]oxazinyl, 2,3-dihydroindolyl, 1,3-dihydroisobenzofuranyl, 2,3-dihydroisoindolyl, 6,7-dihydropyrrolizinyl, dihydroquinolin-2-onyl, dihydroquinolin-4-onyl, furanyl, imidazo[1,2-a]pyrazinyl, imidazo[1,2-a]pyridyl, imidazolyl, imidazopyridyl, imidazo[4,5-d]thiazolyl, indazolyl, indolizinyl, indolyl, isobenzofuranyl, isobenzothienyl, isochromanyl, isochromenyl, isoindoyl, isoquinolin-2-onyl, isoquinolinyl, isothiazolyl, isoxazolyl, naphthyridinyl, 1,2,4-oxadiazoyl, 1,3,4-oxadiazoyl, 1,2,5-oxadiazoyl, oxazolopyridyl, oxazolyl, 2-oxo-2,3-dihydrobenzimidazolyl, 2-oxo-2,3-dihydroindolyl, 1-oxoindanyl, phthalazinyl, pteridinyl, purinyl, pyrazinyl, pyrazolo[1,5-a]pyridyl, pyrazolo[1,5-a]pyrimidinyl, pyrazolyl, pyridazinyl, pyridopyrimidinyl, pyridyl (pyridinyl), pyridyl-N-oxide, pyrimidinyl, pyrimidopyrimidinyl, pyrrolopyridyl, pyrrolopyrimidinyl, pyrrolyl, quinazolinyl, quinolin-4-onyl, quinolinyl, quinoxalinyl, 1,2,3,4-tetrahydroquinolinyl, 1,2,3,4-tetrahydroisoquinolinyl, tetrazolyl, 1,2,4-thiadiazolyl, 1,3,4-thiadiazolyl, 1,2,5-thiadiazolyl, thiazolyl, thieno[2,3-d]imidazolyl, thieno[3,2-b]pyrrolyl, thieno[3,2-b]thiophenyl, thienyl, triazinyl, or triazolyl.

Preferred heteroaryl groups are oxadiazolyl, oxazolyl, isoxazolyl, triazolyl, thiazolyl, thienyl, pyrrolyl, furanyl, pyrazolyl, pyridyl, pyridazinyl, and pyrimidinyl, more preferred is oxadiazolyl, pyrazolyl and pyridyl.

The definition pyrazole includes the isomers 1H-, 3H- and 4H-pyrazole. Preferably pyrazolyl denotes 1H-pyrazolyl.

The definition imidazole includes the isomers 1H-, 2H- and 4H-imidazole. A preferred definition of imidazolyl is 1H-imidazolyl.

The definition triazole includes the isomers 1H-, 3H- and 4H-[1,2,4]-triazole as well as 1H-, 2H- and 4H-[1,2,3]-triazole. The definition triazolyl therefore includes 1H-[1,2,4]-triazol-1-, -3- and -5-yl, 3H[1,2,4]-triazol-3- and -5-yl, 4H[1,2,4]-triazol-3-, -4- and -5-yl, 1H-[1,2,3]-triazol-1-, -4- and -5-yl, 2H[1,2,3]-triazol-2-, -4- and -5-yl as well as 4H[1,2,3]-triazol-4- and -5-yl.

The term tetrazole includes the isomers 1H-, 2H- and 5H-tetrazole. The definition tetrazolyl therefore includes 1H-tetrazol-1- and -5-yl, 2H-tetrazol-2- and -5-yl and 5H-tetrazol-5-yl.

The definition indole includes the isomers 1H- and 3H-indole. The term indolyl preferably denotes 1H-indol-1-yl.

The term isoindole includes the isomers 1H- and 2H-isoindole.

This definition applies for “heteroaryl” in any reasonable context within the present description in the absence of a further definition.

The term “heterocyclyl” within the context of the present invention denotes a saturated or unsaturated but non-aromatic monocyclic 3 to 8 membered, preferably 5-, 6- or 7-membered ring or a 5-12 membered saturated or unsaturated but non-aromatic bicyclic ring system (including spirocyclic and annealed ring systems), which include 1, 2, 3 or 4 heteroatoms, selected from N, O, and/or S, as defined by—S(O)ᵣ— with r being 0, 1 or 2. Preferred are 1, 2, or 3 heteroatoms.

Preferred are saturated heterocyclyl rings with 5, 6, or 7 ring atoms, of which 1 or 2 are heteroatoms and the remaining are C-atoms. Such heterocyclyl groups are addressed as C₅₋₇-heterocyclyl.

Preferred examples for heterocycloalkyl include morpholinyl, piperidinyl, piperazinyl, thiomorpholinyl, oxathianyl, dithianyl, dioxanyl, pyrrolidinyl, tetrahydrofuranyl, dioxolanyl, oxathiolanyl, imidazolidinyl, tetrahydropyranyl, pyrrolinyl, tetrahydrothienyl, oxazolidinyl, homopiperazinyl, homopiperidinyl, homomorpholinyl, homothiomorpholinyl, azetidinyl, 1,3-diazacyclohexanyl or pyrazolidinyl group.

The heterocyclyl group may be bound to the rest of the molecule in more than one way. If no particular bonding arrangement is specified, then all possible arrangements are intended. For example, the term “tetrahydropyranyl” includes 2-, 3-, or 4- tetrahydropyranyl and the like. In cases with more than one ring, the bonding to the rest of the molecule is via at least one ring atom of the ring comprising the at least one heteroatom.

The order of preference of heterocyclic ring systems is: monocyclic ring are more preferred than bicyclic ring systems.

Examples for heterocyclic are the following groups:

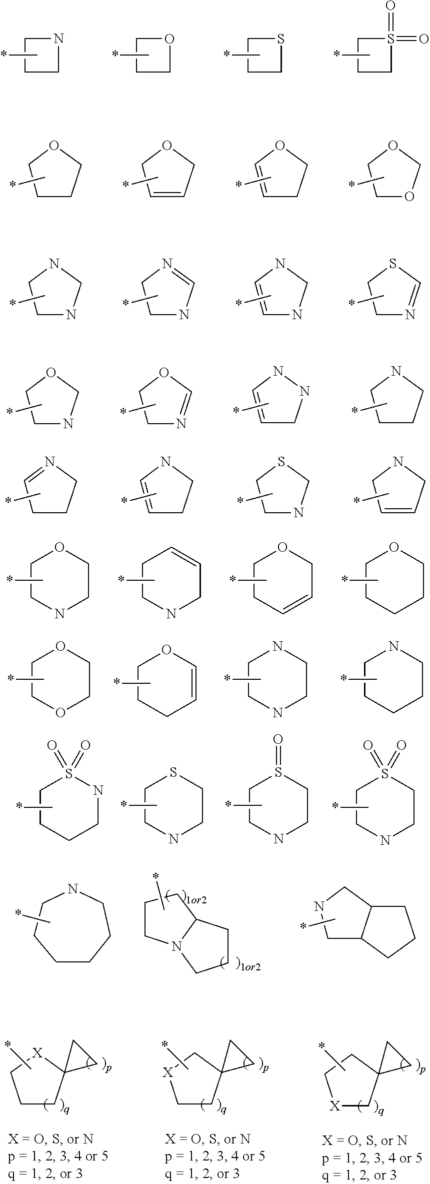

The above definition applies for “heterocyclyl” in any reasonable context within the present description in the absence of a further definition.

The following schemes shall illustrate a process to manufacture the compounds of the present invention by way of example:

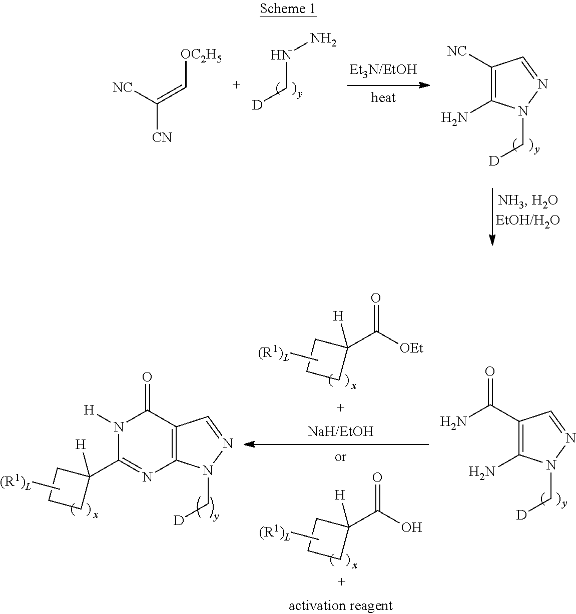

Scheme 1: In a first step 2-ethoxymethylene-malononitrile is condensed with mono-substituted hydrazines by heating in an appropriate solvent like ethanol in the presence of a base (e.g. triethylamine) to form 5-amino-1H-pyrazole-4-carbonitriles. These compounds are converted in a second step to the corresponding amides, e.g. by treatment of an ethanolic solution with ammonia (25% in water) and hydrogen peroxide (35% in water). In a third step, heating with carboxylic esters under basic conditions (e.g sodium hydride in ethanol) or carboxylic acids with an activation reagent (e.g. polyphosphoric acid) leads to pyrazolo[3,4-d]pyrimidin-4-ones as final products [cf., for example, A. Miyashita et al., Heterocycles 1990, 31, 1309ff].

Further alternative processes for preparing pyrazolo[3,4-d]pyrimidin-4-ones are known in the art and can likewise be employed for synthesizing the compounds of the invention (see, for example: P. Schmidt et al., Helvetica Chimica Acta 1962, 189, 1620ff.).

The mono-substituted hydrazine derivatives, that are used in step 1 of scheme 1 can be prepared by reductive amination of a ketone with hydrazinecarboxylic acid tert-butyl ester followed by a deprotection step as shown in scheme 2 [cf., for example, J. W. Timberlake et al., “Chemistry of Hydrazo-,Azo-, and Azoxy Groups”; Patai, S., Ed.; 1975, Chapter 4; S. C. Hung et al., Journal of organic Chemistry 1981, 46, 5413-5414].

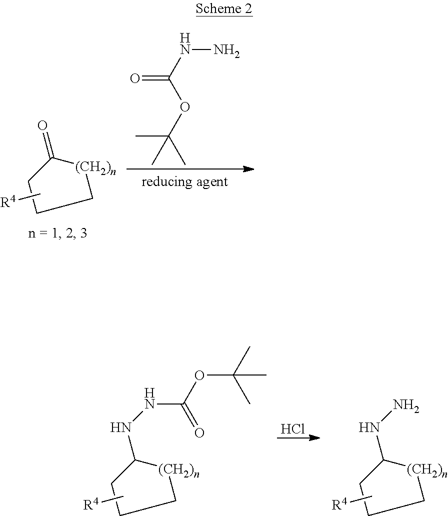

Scheme 3 illustrates as a further example the preparation of compounds of formula I. Di-esters are reacted with 5-amino-1H-pyrazole-4-carboxamides and the intermediate formed is subsequently saponified using aqueous sodium hydroxide. The carboxylic acid formed can be reacted with an amine in an amide coupling-reaction after activation, e.g. by TBTU.

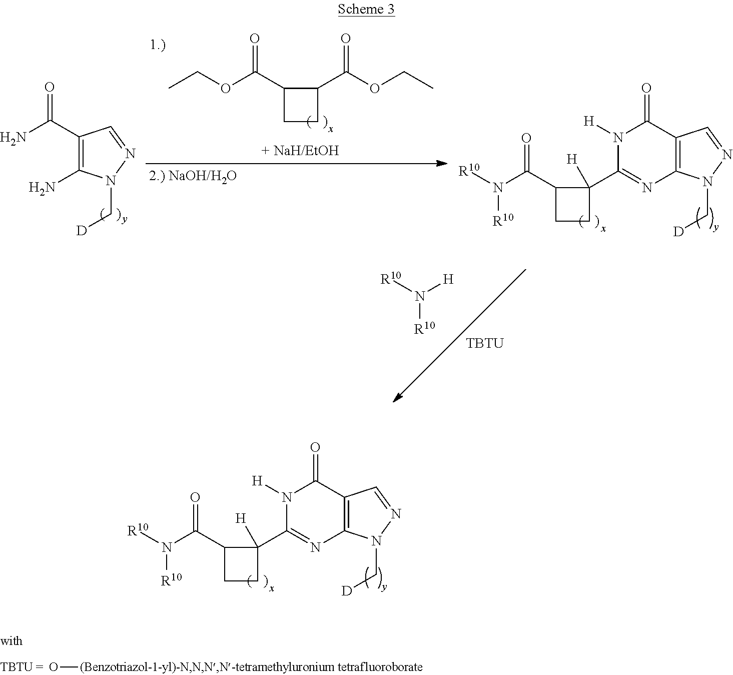

Further information also can be found in WO04099210 (in particular page 9, last paragraph to page 14, line 8, incorporated by reference).

The compounds of the invention show a valuable range of pharmacological effects which could not have been predicted. They are characterised in particular by inhibition of PDE9A.

Preferably the compounds according to the present invention show a high selectivity profile in view of inhibiting or modulating specific members within the PDE9 family or other PDE families, with a clear preference (selectivity) towards PDE9A inhibition.

The compounds of the present invention are supposed to show a favourable safety profile.

### Method of Treament

The present invention refers to compounds, which are considered effective and selective inhibitors of phosphodiesterase 9A and can be used for the development of medicaments. Such medicaments shall preferably be used for the treatment of diseases in which the inhibition of PDE9A can evolve a therapeutic, prophylactic or disease modifying effect. Preferably the medicaments shall be used to improve perception, concentration, cognition, learning or memory, like those occurring in particular in situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic dementia, general concentration impairments, concentration impairments in children with learning and memory problems, Alzheimer's disease, Lewy body dementia, dementia with degeneration of the frontal lobes, including Pick's syndrome, Parkinson's disease, progressive nuclear palsy, dementia with corticobasal degeneration, amyotropic lateral sclerosis (ALS), Huntington's disease, multiple sclerosis, thalamic degeneration, Creutzfeld-Jacob dementia, HIV dementia, schizophrenia with dementia or Korsakoff's psychosis.

Another aspect of the present invention concerns the treatment of a disease which is accessible by PDE9A modulation, in particular sleep disorders like insomnia or narcolepsy, bipolar disorder, metabolic syndrome, obesity, diabetes mellitus, including type 1 or type 2 diabetes, hyperglycemia, dyslipidemia, impaired glucose tolerance, or a disease of the testes, brain, small intestine, skeletal muscle, heart, lung, thymus or spleen.

Thus the medical aspect of the present invention can be summarised in that it is considered that a compound according to formula (I), in particular the compounds of the embodiments as listed in the matrix I or a compound selected from the compounds I through 47 as listed in table 1 is used as a medicament, preferably for humans.

Such a medicament preferably is for the treatment of a CNS disease.

In an alternative use, the medicament is for the treatment of a CNS disease, the treatment of which is accessible by the inhibition of PDE9.

In an alternative use, the medicament is for the treatment of a disease that is accessible by the inhibition of PDE9.

In an alternative use, the medicament is for the treatment, amelioration and/or prevention of cognitive impairment being related to perception, concentration, cognition, learning or memory.

In an alternative use, the medicament is for the treatment amelioration and/or prevention of cognitive impairment being related to age-associated learning and memory impairments, age-associated memory losses, vascular dementia, craniocerebral trauma, stroke, dementia occurring after strokes (post stroke dementia), post-traumatic dementia, general concentration impairments, concentration impairments in children with learning and memory problems, Alzheimer's disease, Lewy body dementia, dementia with degeneration of the frontal lobes, including Pick's syndrome, Parkinson's disease, progressive nuclear palsy, dementia with corticobasal degeneration, amyotropic lateral sclerosis (ALS), Huntington's disease, multiple sclerosis, thalamic degeneration, Creutzfeld-Jacob dementia, HIV dementia, schizophrenia with dementia or Korsakoff's psychosis.

In an alternative use, the medicament is for the treatment of Alzheimer's disease.

In an alternative use, the medicament is for the treatment of sleep disorders, bipolar disorder, metabolic syndrome, obesity, diabetis mellitus, hyperglycemia, dyslipidemia, impaired glucose tolerance, or a disease of the testes, brain, small intestine, skeletal muscle, heart, lung, thymus or spleen.

### Pharmaceutical Compositions

Medicaments for administration comprise a compound according to the present invention in a therapeutically effective amount. By “therapeutically effective amount” it is meant that if the medicament is applied via the appropriate regimen adapted to the patient's condition, the amount of said compound of formula (I) will be sufficient to effectively treat, to prevent or to decelerate the progression of the corresponding disease, or otherwise to ameliorate the estate of a patient suffering from such a disease. It may be the case that the “therapeutically effective amount” in a mono-therapy will differ from the “therapeutically effective amount” in a combination therapy with another medicament.

The dose range of the compounds of general formula (I) applicable per day is usually from 0.1 to 5000 mg, preferably 0.1 to 1000 mg, preferably from 2 to 500 mg, more preferably from 5 to 250 mg, most preferably from 10 to 100 mg. A dosage unit (e.g. a tablet) preferably contains between 2 and 250 mg, particularly preferably between 10 and 100 mg of the compounds according to the invention.

The actual pharmaceutically effective amount or therapeutic dosage will of course depend on factors known by those skilled in the art such as age, weight, gender or other condition of the patient, route of administration, severity of disease, and the like.

The compounds according to the invention may be administered by oral, parenteral (intravenous, intramuscular etc.), intranasal, sublingual, inhalative, intrathecal, topical or rectal route. Suitable preparations for administering the compounds according to the present invention include for example patches, tablets, capsules, pills, pellets, dragees, powders, troches, suppositories, liquid preparations such as solutions, suspensions, emulsions, drops, syrups, elixirs, or gaseous preparations such as aerosols, sprays and the like. The content of the pharmaceutically active compound(s) should be in the range from 0.05 to 90 wt.-%, preferably 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilisers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatin and the like. Lubricants such as magnesium stearate, sodium laurylsulphate and talc may also be used to produce the tablets. In the case of aqueous suspensions the active substances may be combined with various flavour enhancers or colourings in addition to the abovementioned excipients.

The dosage of the compounds according to the invention is naturally highly dependent on the method of administration and the complaint which is being treated. When administered by inhalation the compounds of formula (I) are characterised by a high potency even at doses in the microgram range. The compounds of formula (I) may also be used effectively above the microgram range. The dosage may then be in the gram range, for example.

Combinations with Other Active Substances

In another aspect the present invention relates to the above-mentioned pharmaceutical formulations as such which are characterised in that they contain a compound according to the present invention.

A further aspect of the present invention refers to a combination of each of the compounds of the present invention, preferably at least one compound according to the present invention with another compound selected from the group of for example beta-secretase inhibitors; gamma-secretase inhibitors; gamma-secretase modulators; amyloid aggregation inhibitors such as e.g. alzhemed; directly or indirectly acting neuroprotective substances, such as e.g. dimebon; directly or indirectly acting disease-modifying substances; anti-oxidants, such as e.g. vitamin E; ginko biloba or ginkolide; anti-inflammatory substances, such as e.g. Cox inhibitors, NSAIDs additionally or exclusively having Aβ lowering properties; HMG-CoA reductase inhibitors such as statins; acetylcholine esterase inhibitors, such as donepezil, rivastigmine, tacrine, galantamine; NMDA receptor antagonists such as e.g. memantine; AMPA receptor agonists; AMPA receptor positive modulators, AMPkines; glycine transporter 1 inhibitors; monoamine receptor reuptake inhibitors; substances modulating the concentration or release of neurotransmitters; substances inducing the secretion of growth hormone such as ibutamoren mesylate and capromorelin; CB-1 receptor antagonists or inverse agonists; antibiotics such as minocyclin or rifampicin; PDE1, PDE2, PDE4, PDE5 and/or PDE10 inhibitors; GABAA receptor inverse agonists or GABAA receptor antagonists; nicotinic receptor agonists, partial agonists or positive modulators; alpha4beta2 nicotinic receptor agonists, partial agonists or positive modulators; alpha7 nicotinic receptor agonists, partial agonists or positive modulators; histamine receptor H3 antagonists; 5-HT4 receptor agonists, partial agonists or positive modulators; 5-HT6 receptor antagonists; alpha2-adrenoreceptor antagonists; calcium antagonists; muscarinic receptor M1 agonists, partial agonists or positive modulators; muscarinic receptor M2 antagonists; muscarinic receptor M4 antagonists; metabotropic glutamate receptor 5 positive modulators; metabotropic glutamate receptor 2 antagonists; and other substances that modulate receptors or enzymes in a manner such that the efficacy and/or safety of the compounds according to the invention is increased and/or unwanted side effects are reduced.

This invention further relates to pharmaceutical compositions containing one or more, preferably one active substance, which is selected from the compounds according to the invention and/or the corresponding salts, as well as one or more, preferably one active substance selected from among alzhemed, vitamin E, ginkolide, donepezil, rivastigmine, tacrine, galantamine, memantine, ibutamoren mesylate, capromorelin, minocyclin and/or rifampicin, optionally together with one or more inert carriers and/or diluents.

The compounds according to the invention may also be used in combination with immunotherapies such as e.g. active immunisation with Abeta or parts thereof or passive immunisation with humanised anti-Abeta antibodies or antibody fragments for the treatment of the above-mentioned diseases and conditions.

The combinations according to the present invention may be provided simultaneously in one and the same dosage form, i.e. in form of a combination preparation, for example the two components may be incorporated in one tablet, e.g. in different layers of said tablet. The combination may be also provided separately, in form of a free combination, i.e the compounds of the present invention are provided in one dosage form and one or more of the above mentioned combination partners is provided in another dosage form. These two dosage forms may be equal dosage forms, for example a co-administration of two tablets, one containing a therapeutically effective amount of the compound of the present invention and one containing a therapeutically effective amount of the above mentioned combination partner. It is also possible to combine different administration forms, if desired. Any type of suitable administration forms may be provided.

The compound according to the invention, or a physiologically acceptable salt thereof, in combination with another active substance may be used simultaneously or at staggered times, but particularly close together in time. If administered simultaneously, the two active substances are given to the patient together; if administered at staggered times the two active substances are given to the patient successively within a period of less than or equal to 12, particularly less than or equal to 6 hours.

The dosage or administration forms are not limited, in the frame of the present invention any suitable dosage form may be used. Exemplarily the dosage forms may be selected from solid preparations such as patches, tablets, capsules, pills, pellets, dragees, powders, troches, suppositories, liquid preparations such as solutions, suspensions, emulsions, drops, syrups, elixirs, or gaseous preparations such as aerosols, sprays and the like.

The dosage forms are advantageously formulated in dosage units, each dosage unit being adapted to supply a single dose of each active component being present. Depending from the administration route and dosage form the ingredients are selected accordingly.

The dosage for the above-mentioned combination partners is expediently ⅕ of the normally recommended lowest dose up to 1/1 of the normally recommended dose.

The dosage forms are administered to the patient for example 1, 2, 3, or 4 times daily depending on the nature of the formulation. In case of retarding or extended release formulations or other pharmaceutical formulations, the same may be applied differently (e.g. once weekly or monthly etc.). It is preferred that the compounds of the invention be administered either three or fewer times, more preferably once or twice daily.

### EXAMPLES

### Pharmaceutical Compositions

The following examples propose pharmaceutical formulations that may illustrate the present invention without restricting its scope:

The term “active substance” denotes one or more compounds according to the invention including the salts thereof.

### Example A

### Tablets Containing 100 Mg of Active Substance

### Composition:

1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

### Example B

### Tablets Containing 150 Mg of Active Substance

### Composition:

1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

### Example C

### Hard Gelatine Capsules Containing 150 mg of Active Substance

1 capsule contains:

| | | |
|---|---|---|
| active substance | 150.0 | mg |
| corn starch (dried) | approx. 80.0 | mg |
| lactose (powdered) | approx. 87.0 | mg |
| magnesium stearate | 3.0 | mg |
| | approx. 320.0 | mg |

Capsule shell: size 1 hard gelatine capsule.

### Example D

### Suppositories Containing 150 mg of Active Substance

1 suppository contains:

| | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2,000.0 | mg |

### Example E

### Ampoules Containing 10 Mg Active Substance

### Composition:

| active substance | 10.0 | mg |
|---|---|---|
| 0.01N hydrochloric acid | q.s. | |
| double-distilled water | ad 2.0 | mL |

### Example F

### Ampoules Containing 50 Mg of Active Substance

### Composition:

| active substance | 50.0 | mg |
|---|---|---|
| 0.01N hydrochloric acid | q.s. | |
| double-distilled water | ad 10.0 | mL |

The preparation of any the above mentioned formulations can be done following standard procedures.

### Biological Assay

The in vitro effect of the compounds of the invention can be shown with the following biological assays.

### PDE9A2 Assay Protocol:

The PDE9A2 enzymatic activity assay was run as scintillation proximity assay (SPA), in general according to the protocol of the manufacturer (Amersham Biosciences, product number: TRKQ 7100).

As enzyme source, lysate (PBS with 1% Triton X-100 supplemented with protease inhibitors, cell debris removed by centrifugation at 13.000 rpm for 30 min) of SF 9 cell expressing the human PDE9A2 was used. The total protein amount included in the assay varied upon infection and production efficacy of the SF9 cells and lay in the range of 0.1-100 ng.

In general, the assay conditions were as follows:
total assay volume: 40 microliter
protein amount: 0.1-50 ng
substrate concentration (cGMP): 20 nanomolar; ˜1 mCi/l
incubation time: 60 min at room temperature
final DMSO concentration: 0.2-1%

The assays were run in 384-well format. The test reagents as well as the enzyme and the substrate were diluted in assay buffer. The assay buffer contained 50 mM Tris, 8.3 mM MgCl2, 1.7 mM EGTA, 0.1% BSA, 0.05% Tween 20; the pH of assay buffer was adjusted to 7.5. The reaction was stopped by applying a PDE9 specific inhibitor (e.g. compounds according to WO04099210 or WO04099211) in excess.

### Determination of % Inhibition:

The activity of the positive control (minus the negative control=background) is set to 100% and activity in the presence of test compound is expressed relative to these 100%. Within this setting, an inhibition above 100% might be possible due to the nature of the variation of the positive control within the assay, however, in this case the reported % inhibition had been adjusted to 100%.

### Determination of IC50:

1050 can be calculated with GraphPadPrism or other suited software setting the positive control as 100 and the negative control as 0. For calculation of 1050 dilutions of the test compounds (substrates) are to be selected and tested following the aforementioned protocol.

### Data

In the following, % inhibition data will illustrate that the compounds according to the present invention are suited to inhibit PDE9 and thus provide useful pharmacological properties. The examples are not meant to be limiting. The table also provides IC₅₀ values. The values are presented as being within a nanomolar range (nM), i.e. within the range of either 1 nanomolar to 200 nanomolar or within the range of 201 nanomolar to 5000 nanomolar. The specific IC₅₀ value is within said range. The example number refer to the final examples as outlined in the section Exemplary embodiments.

All data are measured according to the procedure described herein.

| | | IC₅₀ within |
|---|---|---|
| | | range |
| Example | % inhibition | [nanomolar |
| No. | of PDE9A2* | (nM)] |
| 1 | 99 | 1-200 |
| 2 | 100 | 1-200 |
| 3 | 100 | 1-200 |
| 4 | 97 | 1-200 |
| 5 | 100 | 1-200 |
| 6 | 101 | 1-200 |
| 7 | 102 | 1-200 |
| 8 | 100 | 1-200 |
| 9 | 100 | 1-200 |
| 10 | 97 | 1-200 |
| 11 | 94 | 1-200 |
| 12 | 99 | 1-200 |
| 13 | 97 | 1-200 |
| 14 | 100 | 1-200 |
| 15 | 101 | 1-200 |
| 16 | 98 | 1-200 |
| 17 | 99 | 1-200 |
| 18 | 96 | 1-200 |
| 19 | 92 | 1-200 |
| 20 | 71 | 201-5000 |
| 21 | 66 | 1-200 |
| 22 | 90 | 1-200 |
| 23 | 92 | 201-5000 |
| 24 | 101 | 1-200 |
| 25 | 95 | 201-5000 |
| 26 | 99 | 1-200 |
| 27 | 98 | 1-200 |
| 28 | 94 | 1-200 |
| 29 | 98 | 1-200 |
| 30 | 96 | 1-200 |
| 31 | 80 | 201-5000 |
| 32 | 91 | 1-200 |
| 33 | 83 | 201-5000 |
| 34 | 57 | 201-5000 |
| 35 | 50 | 201-5000 |
| 36 | 90 | 1-200 |
| 37 | — | 1-200 |
| 38 | — | 1-200 |
| 39 | — | 201-5000 |
| 40 | — | 201-5000 |
| 41 | — | 201-5000 |
| 42 | — | 201-5000 |
| 43 | — | 201-5000 |
| 44 | — | 201-5000 |
| 45 | — | 201-5000 |
| 46 | 91 | 1-200 |
| 47 | 89 | 201-5000 |

*at 10 micromolar concentration

### In Vivo Effect:

The in vivo effect of the compounds of this invention can be tested in the Novel Object Recognition test according to the procedure of Prickaerts et al.

### (Neuroscience, 2002, 113, 351-361).

For further information concerning biological testing of the compounds of the present invention see also Neuropharmacology, 2008, 55, 908-918.

### Chemical Manufacture

### Abbreviations:

CDI 1,1′-carbonyldiimidazole
DIPEA diisopropylethylamine
DME 1,2-dimethoxyethan
DMF dimethylformamide
ESI electrospray ionization (in MS)
Exp. example
h hour(s)
HATU O-(7-azabenzotriazol-1-yl)-N,N,N′,N′-tetramethyluronium hexafluorophosphate
HPLC high performance liquid chromatography
HPLC-MS coupled high performance liquid chromatography-mass spectrometry
M molar (mol/L)
min minutes
MS mass spectrometry
NMP 1-methyl-2-pyrrolidinone
Rₜ retention time (in HPLC)
TBTU O-(benzotriazol-1-yl)-N,N,N′,N′-tetramethyluronium tetrafluoroborate
TFA trifluoroacetic acid
THF tetrahydrofuran
TLC thin-layer chromatography
LC-MS methods:

### Method 1

MS apparatus type: Waters Micromass ZQ; HPLC apparatus type: Waters Alliance 2695, Waters 2996 diode array detector; column: Varian Microsorb 100 C18, 30×4.6 mm, 3.0 μm; eluent A: water+0.13% TFA, eluent B: acetonitrile; gradient: 0.0 min 5% B→0.18 min 5% B→2.0 min 98% B→2.2 min 98% B→2.3 min 5% B→2.5 min 5% B; flow rate: 3.5 mL/min; UV detection: 210-380 nm.

### Method 2

MS apparatus type: Waters Micromass ZQ; HPLC apparatus type: Waters Alliance 2695, Waters 2996 diode array detector; column: Varian Microsorb 100 C18, 30×4.6 mm, 3.0 μm; eluent A: water+0.13% TFA, eluent B: methanol; gradient: 0.0 min 5% B→0.35 min 5% B→3.95 min 100% B→4.45 min 100% B→4.55 min 5% B→4.9 min 5% B; flow rate: 2.4 mL/min; UV detection: 210-380 nm.

### Microwave Heating:

### Microwave Apparatus Types:

Discover® CEM instruments, equipped with 10 and 35 mL vessels;
Biotage Initiator Sixty.

### General Comment Concerning the Presentation of the Structures

Some compounds have one or more chiral centres. The depicted structure will not necessarily show all the possible stereochemical realisations of the compound but only one. However, in such cases a term like “cis-racemic mixture” is added next to the depicted structure in order to pin point to the other stereochemical options.

An example is given for Example 5A, below. The presented structural formula is

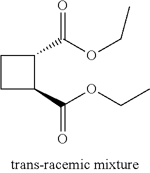

The added term “trans-racemic mixture” points to the second stereochemical option:

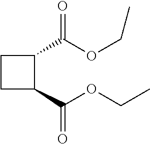

This principle applies to other depicted structures as well.

### Starting Compounds

### Example 1A

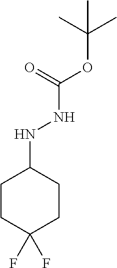

5.00 g (37.3 mmol) 4,4-difluorocyclohexanone were mixed with 200 mL isopropanol and 5.30 g (40.1 mmol) t-butylcarbazate; 0.75 mL conc. acetic acid and PtO₂ were added. The reaction mixture was hydrogenated at room temperature (12 h, 50 psi). The reaction mixture was filtered and the solvent was evaporated under reduced pressure. 10 g (98%) of the product were obtained.

MS (ESI pos): m/z=251 (M+H)⁺

### Example 2A

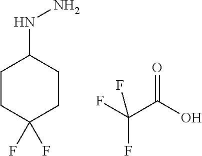

4.00 g (16.0 mmol) of Example 1A were mixed with 40 mL dichloromethane and 5.50 mL (71.4 mmol) trifluoroacetic acid were added. The reaction mixture was stirred 12 h at room temperature. The solvent was evaporated under reduced pressure. 4.0 g (95%) of the product were obtained.

MS (ESI pos): m/z=151 (M+H)⁺

### Example 3A

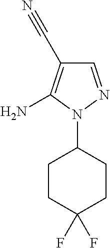

4.20 g (16.0 mmol) of Example 2A were suspended with 2.15 g (17.6 mmol) of ethoxymethylenemalononitrile in 50 mL of ethanol and 6.70 mL (48.0 mmol) of triethylamine were added. The reaction mixture was heated to 50° C. for 2 h. After cooling to room temperature the solvent was removed under reduced pressure. The residue was suspended in dichloromethane. The suspension was filtered. 3.9 g (96%) of the product were obtained.

HPLC-MS (Method 1): Rₜ=1.19 min

MS (ESI pos): m/z=225 (M−H)⁻

### Example 4A

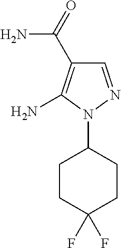

3.88 g (14.6 mmol) of Example 3A were mixed with 40 mL of ethanol. At room temperature a solution of 35 mL (0.41 mol) hydrogen peroxide (35% in water) in 20 mL ammonia (25% in water) was added over a period of 10 min. The reaction mixture was stirred at room temperature for 2 h. The solution was concentrated to a volume of 50 mL under reduced pressure. The residue was mixed with dichloromethane and water. The organic layer was extracted with water and 40% Na₂S₂O₃ solution. The organic layer was dried, filtered and the filtrate was concentrated under reduced pressure. 2.44 g (68%) of the product were obtained.

HPLC-MS (Method 1): Rₜ=0.91 min

MS (ESI pos): m/z=245 (M+H)⁺

### Example 5A

### Trans-Racemic Mixture

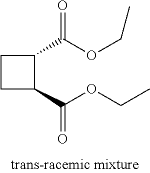

2.00 g (13.9 mmol) trans-cyclobutan-1,2-dicarboxylic acid were mixed with 16 mL ethanol at 0° C. and 2.21 ml (30.5 mmol) thionylchloride were slowly added. The mixture was allowed to warm to room temperature and stirred for 1 hour. The solvent was removed under reduced pressure and the product was filtered through a pad of activated basic alumina. 2.71 g (98%) of the product were obtained.

HPLC-MS (Method 1): Rₜ=1.34 min

MS (ESI pos): m/z=201 (M+H)⁺

The following examples were synthesized in analogy to the preparation of Example 5A, using the corresponding dicarboxylic acids as starting materials:

| | | | | MS |
|---|---|---|---|---|
| | | starting | | (ESI pos, |
| Example | structure | material | Rₜ [min] | m/z) |
| Exp. 5B cis-racemic mixture | 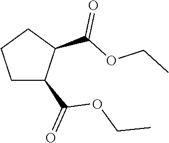 | 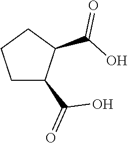 | 1.39 (Method 1) | 215 (M + H)⁺ |
| Exp. 5C trans-racemic mixture | 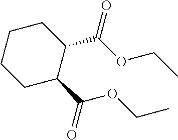 | 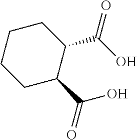 | 1.55 (Method 1) | 229 (M + H)⁺ |

### Example 6A

### Trans-Racemic Mixture

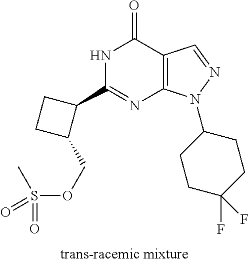

100 mg (0.30 mmol) of Example 33 were mixed with 1 mL of dichloromethane and 60 μL (0.44 mmol) 2,4,6-collidine at 0° C. A solution of 304 (0.38 mmol) of methanesulfonylchloride in 1 mL of dichloromethane was added dropwise. The reaction mixture was heated to room temperature and stirred for 2 h. Saturated sodium hydrogen carbonate solution was added and the phases were separated. The organic layer was dried and the solvent was removed under reduced pressure. 121 mg (98%) of the product were obtained. The material was used for the next reaction without further purification.

HPLC-MS (Method 1): Rₜ=1.37 min

MS (ESI pos): m/z=417 (M+H)⁺

### Example 7A

### Trans-Racemic Mixture

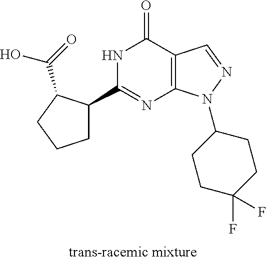

150 mg (0.61 mmol) of Example 4A were mixed with 2 mL of absolute ethanol, 395 mg (1.84 mmol) of Example 5B and 118 mg (2.95 mmol) of sodium hydride (60% suspension in mineral oil) were added. The reaction mixture was heated to 140° C. for 30 min in a microwave oven. The mixture was cooled to room temperature and sodium hydroxide solution (4 M in water) was added. The solvent was removed under reduced pressure. The substance was purified by preparative HPLC (eluent A: water+0.13% TFA, eluent B: acetonitrile). 109 mg (48%) of the product were obtained.

HPLC-MS (Method 1): Rₜ=1.29 min

MS (ESI pos): m/z=367 (M+H)⁺

The following examples were synthesized in analogy to the preparation of Example 7A, using the corresponding dicarboxylic esters as starting materials:

| | | | | MS |
|---|---|---|---|---|
| | | starting | Rₜ | (ESI |
| Example | structure | material | [min] | pos, m/z) |
| Exp. 7B trans- racemic mixture | 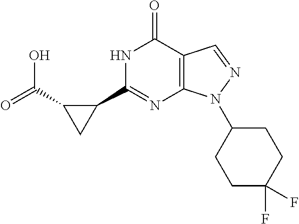 | 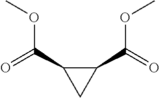 | 1.22 (Method 1) | 339 (M + H)⁺ |
| Exp. 7C trans- racemic mixture | 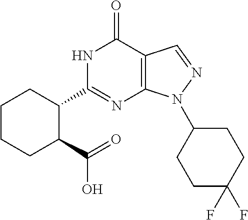 | Exp. 5C | 1.31 (Method 1) | 381 (M + H)⁺ |

### Example 8A

### Trans-Racemic Mixture

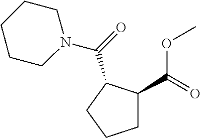

300 mg (1.74 mmol) trans-cyclopentane-1,2-dicarboxylic acid monomethyl ester were mixed with 2 mL DMF and 0.30 mL (1.74 mmol) DIPEA. 615 mg (1.92 mmol) TBTU was added and stirred for 10 minutes at room temperature. 300 μL (1.74 mmol) DIPEA and 290 μL piperidine were added and stirred at room temperature for 1 h. The reaction mixture was evaporated under reduced pressure. The residue was purified by preparative HPLC (eluent A: water+0.13% TFA, eluent B: acetonitrile). 400 mg (96%) of the product were obtained.

HPLC-MS (Method 1): Rₜ=1.26 min

MS (ESI pos): m/z=240 (M+H)⁺

### Example 9A

### Trans-Racemic Mixture

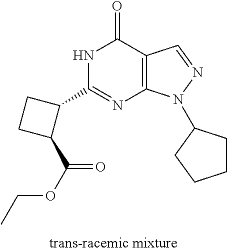

0.20 g (1.03 mmol) of 5-amino-1-cyclopentyl-1H-pyrazole-4-carboxylic acid amide (DE 10238724) were mixed with 2 mL of absolute ethanol, 0.62 g (3.09 mmol) of Example 5A, and 0.16 g (4.12 mmol) of sodium hydride (60% suspension in mineral oil) were added. The reaction mixture was heated to 140° C. for 30 min in a microwave oven. The mixture was cooled to room temperature and the solvent was evaporated under reduced pressure. The substance was purified by preparative HPLC (eluent A: water+0.13% TFA, eluent B: acetonitrile). 87 mg (26%) of the product were obtained.

HPLC-MS (Method 1): Rₜ=1.49 min

MS (ESI pos): m/z=331 (M+H)⁺

### Example 10A

### Trans-Racemic Mixture

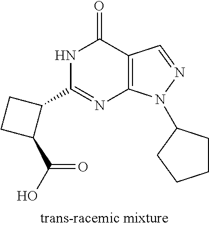

65 mg (0.20 mmol) of Example 9A were mixed with 3 mL methylene chloride and 10 mL sodium hydroxide solution (4 M in water) were added. The mixture was stirred at room temperature for 1 h. The mixture was acidified with hydrochloric acid and extracted with ethyl acetate. The combined organic layers were dried and evaporated under reduced pressure. 57.0 mg (96%) of the product were obtained.

HPLC-MS (Method 1): Rₜ=1.23 min

MS (ESI pos): m/z=303 (M+H)⁺

The following example was synthesized in analogy to the preparation of Example 10A, using the corresponding esters as starting materials:

| | | starting |
|---|---|---|
| Example | structure | material |
| Exp. 10B mixture of stereoisomers | 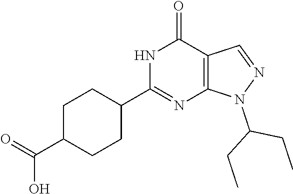 | Exp. 14A |

### Example 11A

### Cis-Racemic Mixture

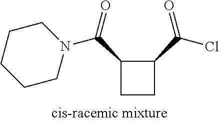

110 mg (0.52 mmol) of cis-2-(piperidine-1-carbonyl)-cyclobutanecarboxylic acid (P. Schenone et al., II Farmaco, Ed. Sc.; 27, 1972, 200-207) were mixed with 1 mL chloroform. 0.08 mL (1.15 mmol) of thionyl chloride were added. The reaction mixture was stirred at reflux for 2 h. The reaction mixture was evaporated under reduced pressure. The residue was used for the next step without further purification.

### Example 12A

### Cis-Racemic Mixture

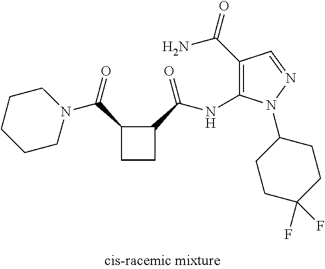

50.0 mg (0.20 mmol) of Example 4A and 119 mg (0.52 mmol) of Example 11A were mixed with 2 mL of NMP were stirred over night at room temperature. The reaction mixture was diluted with water and purified by preparative HPLC (eluent A: water+0.13% TFA, eluent B: acetonitrile). 33 mg (37%) of the product were obtained.

HPLC-MS (Method 1): Rₜ=1.21 min

MS (ESI neg): m/z=436 (M−H)⁻

### Example 13A

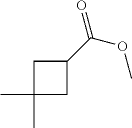

1.25 g (9.75 mmol) 3,3-Dimethylcyclobutanecarboxylic acid were dissolved in 10 mL acetonitrile/methanol (9/1). 6.34 mL Trimethylsilyldiazomethane (2M solution in diethylether) were added. Some drops of acetic acid were added. The reaction mixture was concentrated under reduced pressure. 1.30 g (94%) of the product were obtained.

HPLC-MS (Method 1): Rₜ=1.50 min

MS (ESI neg): m/z=143 (M+H)⁺

The following example was synthesized in analogy to the preparation of Example 13A, using the corresponding dicarboxylic esters as starting materials:

| | | | | MS (ESI |
|---|---|---|---|---|
| Exam- | | | | pos/neg, |
| ple | structure | starting material | Rₜ [min] | m/z) |
| Exp. 13B | 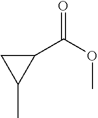 | 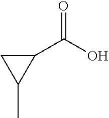 | 1.08 (Method 1) | 113 (M − H)⁻ |

### Example 14A

### Mixture of Stereoisomers

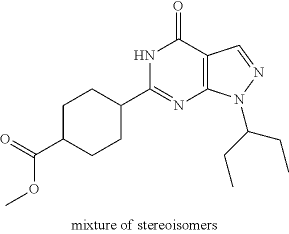

1.00 g (5.10 mmol) of 5-amino-1-(1-ethylpropyl)-1H-pyrazole-4-carboxamide (DE 10238722) was mixed with 3 mL of absolute ethanol, 4.59 g (22.9 mmol) of cyclohexane-1,4-dicarboxylic acid dimethyl ester, and 1.73 g (25.4 mmol) of sodium ethylate were added. The reaction mixture was heated to 78° C. for 12 h. The mixture was cooled to room temperature. Water was added and the mixture was extracted with ethyl acetate. The solvent was removed under reduced pressure. The substance was purified by preparative HPLC (eluent A: water+0.13% TFA, eluent B: acetonitrile). 0.60 g (34%) of the product were obtained.

HPLC-MS: Identity and purity confirmed

NMR: Identity confirmed

### Exemplary Embodiments

### Example 1

### Trans-Racemic Mixture

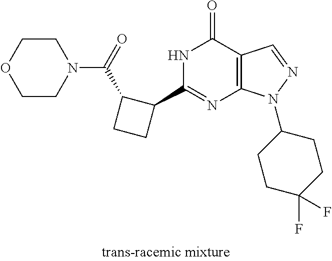

50.0 mg (0.14 mmol) of Example 46 were mixed with 2 mL DMF and 25 μL (0.14 mmol) DIPEA. 59.6 mg (0.16 mmol) HATU were added and stirred for 10 minutes at room temperature. 75 μL (0.43 mmol) DIPEA and 24.6 μL (0.28 mmol) morpholine were added and stirred at room temperature for 1 h. The reaction mixture was evaporated under reduced pressure. The residue was purified by preparative HPLC (eluent A: water+0.13% TFA, eluent B: acetonitrile). 38.7 mg (60%) of the product were obtained.

HPLC-MS (Method 1): Rₜ=1.21 min

MS (ESI pos): m/z=422 (M+H)⁺

The following examples were synthesized in analogy to the preparation of Example 1, using the corresponding pyrazoles and amines as starting materials

| | | | | | MS |
|---|---|---|---|---|---|
| | | starting | starting | | (ESI |
| | | material; | material | | pos, |
| Example | structure | pyrazol | amine | Rₜ [min] | m/z) |
| Exp. 2 diastereomeric mixture; trans configuration at the cyclobutane ring; stereocentre at pyrrolidine is “R” for both diastereomers | 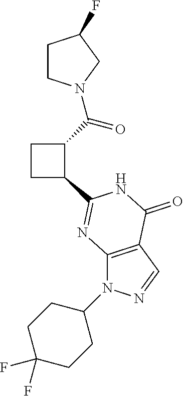 | Exp. 46 | 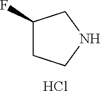 | 1.30 (Method 1) | 424 (M + H)⁺ |
| Exp. 3 trans- racemic mixture | 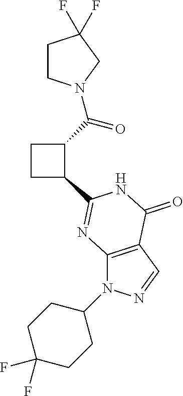 | Exp. 46 | 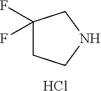 | 1.34 (Method 1) | 442 (M + H)⁺ |
| Exp. 4 trans- racemic mixture | 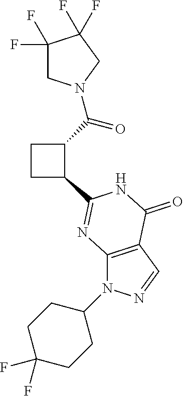 | Exp. 46 | 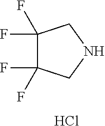 | 1.42 (Method 1) | 478 (M + H)⁺ |
| Exp. 5 trans- racemic mixture | 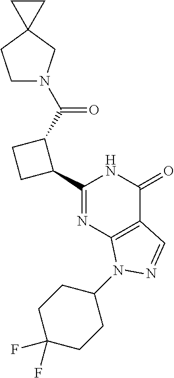 | Exp. 46 | 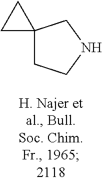 | 1.46 (Method 1) | 432 (M + H)⁺ |
| Exp. 6 trans- racemic mixture | 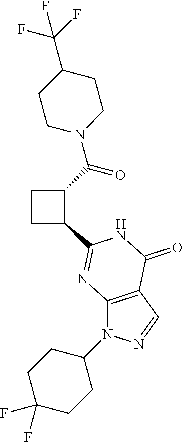 | Exp. 46 | 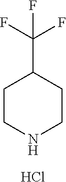 | 1.52 (Method 1) | 488 (M + H)⁺ |
| Exp. 7 diastereomeric mixture; trans configuration at the cyclo- butane ring; stereo- centre at pyrrolidine is “R” for both diastereo- mers | 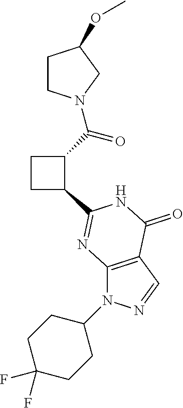 | Exp. 46 | 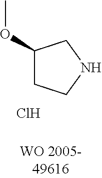 | 1.29 (Method 1) | 436 (M + H)⁺ |
| Exp. 8 diastereomeric mixture; trans configuration at the cyclobutane ring | 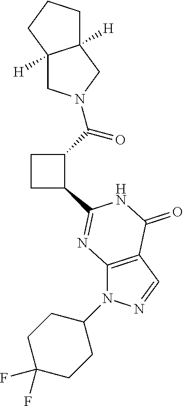 | Exp. 46 | 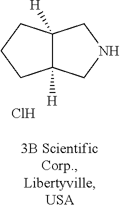 | 1.48 (Method 1) | 446 (M + H)⁺ |
| Exp. 9 trans- racemic mixture | 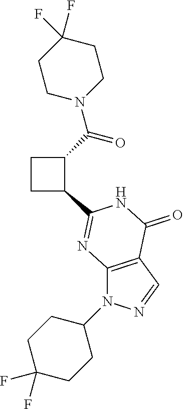 | Exp. 46 | 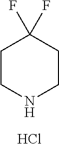 | 1.44 (Method 1) | 456 (M + H)⁺ |
| Exp. 10 trans- racemic mixture | 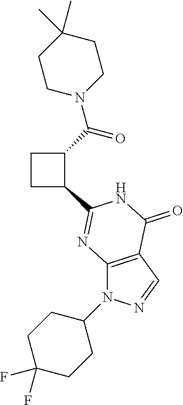 | Exp. 46 | 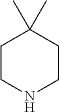 | 1.57 (Method 1) | 448 (M + H)⁺ |
| Exp. 11 trans- racemic mixture | 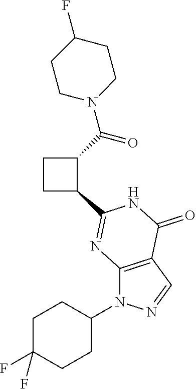 | Exp. 46 | 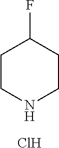 | 1.33 (Method 1) | 438 (M + H)⁺ |
| Exp. 12 trans- racemic mixture | 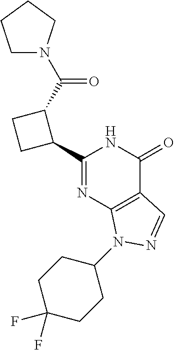 | Exp. 46 | 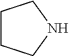 | 1.32 (Method 1) | 406 (M + H)⁺ |
| Exp. 13 trans- racemic mixture | 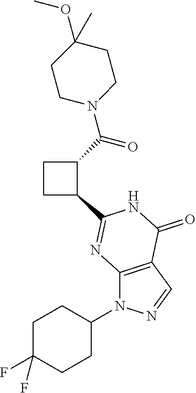 | Exp. 46 | 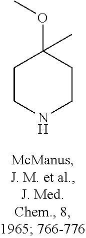 | 1.39 (Method 1) | 464 (M + H)⁺ |
| Exp. 14 trans- racemic mixture | 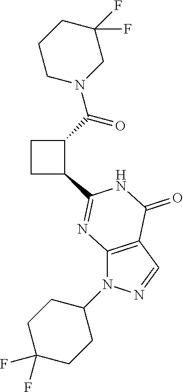 | Exp. 46 | 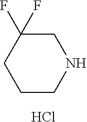 | 1.38 (Method 1) | 456 (M + H)⁺ |
| Exp. 15 trans- racemic mixture | 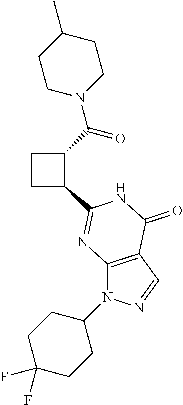 | Exp. 46 | 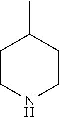 | 1.49 (Method 1) | 434 (M + H)⁺ |
| Exp. 16 trans- racemic mixture | 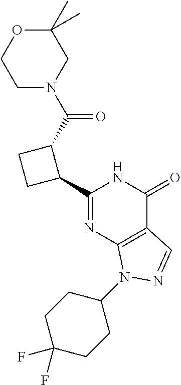 | Exp. 46 | 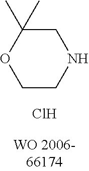 | 1.33 (Method 1) | 450 (M + H)⁺ |
| Exp. 17 trans- racemic mixture | 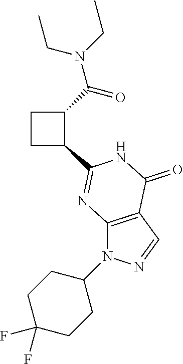 | Exp. 46 | 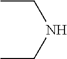 | 1.38 (Method 1) | 408 (M + H)⁺ |
| Exp. 18 trans- racemic mixture | 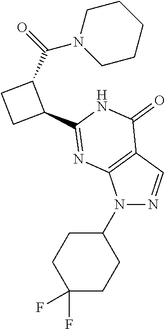 | Exp. 46 | 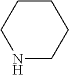 | 1.47 (Method 1) | 420 (M + H)⁺ |
| Exp. 19 trans- racemic mixture | 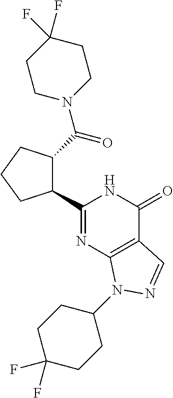 | Exp. 7A | 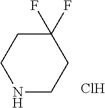 | 1.46 (Method 1) | 470 (M + H)⁺ |
| Exp. 20 trans- racemic mixture | 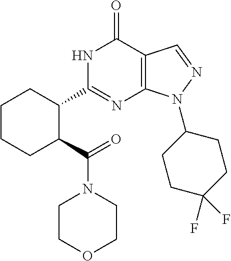 | Exp. 7C | 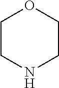 | 1.32 (Method 1) | 450 (M + H)⁺ |
| Exp. 21 trans- racemic mixture | 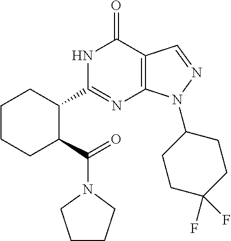 | Exp. 7C | 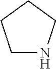 | 1.42 (Method 1) | 434 (M + H)⁺ |
| Exp. 22 trans- racemic mixture | 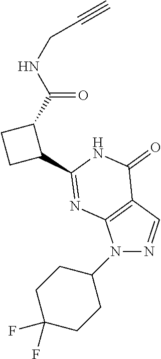 | Exp. 46 | 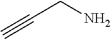 | 2.75 (Method 2) | 390 (M + H)⁺ |

### Example 23

### Trans-Racemic Mixture

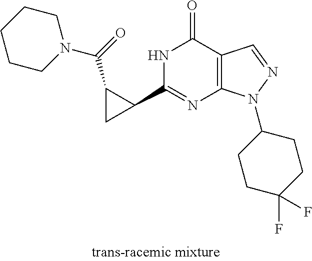

70 mg (0.21 mmol) of Example 7B were mixed with 1 mL DMF and 35 μL (0.21 mmol) DIPEA. 73 mg (0.23 mmol) TBTU were added and stirred for 10 minutes at room temperature. 35 μL (0.21 mmol) DIPEA and 30 μL piperidine were added and stirred at room temperature for 1 h. The reaction mixture was evaporated under reduced pressure. The residue was purified by preparative HPLC (eluent A: water+0.13% TFA, eluent B: acetonitrile). 59 mg (70%) of the product were obtained.

HPLC-MS (Method 1): Rₜ=1.39 min

MS (ESI pos): m/z=406 (M+H)⁺

The following example was synthesized in analogy to the preparation of Example 23:

| | | starting | starting | | |
|---|---|---|---|---|---|
| | | material | material | | MS |
| Example | structure | 1 | 2 | Rₜ [min] | (ESI pos, m/z) |
| Exp. 24 trans- racemic mixture | 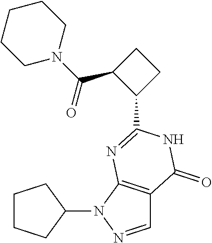 | Exp. 10A | 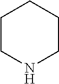 | 1.50 (Method 1) | 370 (M + H)⁺ |

### Example 25

### Trans-Racemic Mixture

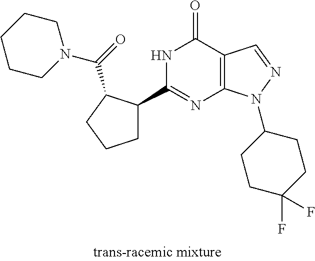

100 mg (0.41 mmol) of Example 4A were mixed with 2 mL of absolute ethanol, 220 mg (0.96 mmol) of Example 8A, and 66.0 mg (1.64 mmol) of sodium hydride (60% suspension in mineral oil) were added. The reaction mixture was heated to 140° C. for 30 min in a microwave oven. The mixture was cooled to room temperature. The solvent was removed under reduced pressure. The substance was purified by preparative HPLC (eluent A: water+0.13% TFA, eluent B: acetonitrile). 12 mg (7%) of the product were obtained.

HPLC-MS (Method 1): Rₜ=1.47 min

MS (ESI pos): m/z=434 (M+H)⁺

The following example was synthesized in analogy to the preparation of Example 25:

| | | starting | starting | | |
|---|---|---|---|---|---|
| | | material | material | | MS |
| Example | structure | 1 | 2 | Rₜ [min] | (ESI pos, m/z) |
| Exp. 26 trans- racemic mixture | 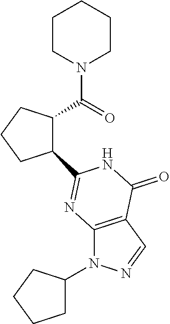 | 5-Amino-1- cyclopentyl- 1H-pyrazole- 4-carboxylic acid amide (DE 10238724) | Exp. 8A | 1.50 (Method 1) | 384 (M + H)⁺ |

### Example 27

### Trans-Racemic Mixture

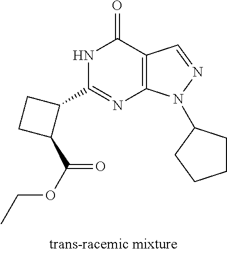

0.20 g (1.03 mmol) of 5-amino-1-cyclopentyl-1H-pyrazole-4-carboxylic acid amide (DE 10238724) were mixed with 2 mL of absolute ethanol, 0.62 g (3.09 mmol) of Example 5A, and 0.16 g (4.12 mmol) of sodium hydride (60% suspension in mineral oil) were added. The reaction mixture was heated to 140° C. for 30 min in a microwave oven. The mixture was cooled to room temperature and the solvent was evaporated under reduced pressure. The substance was purified by preparative HPLC (eluent A: water+0.13% TFA, eluent B: acetonitrile). 87.0 mg (26%) of the product were obtained.

HPLC-MS (Method 1): Rₜ=1.49 min

MS (ESI pos): m/z=331 (M+H)⁺

### Example 28

### Trans-Racemic Mixture

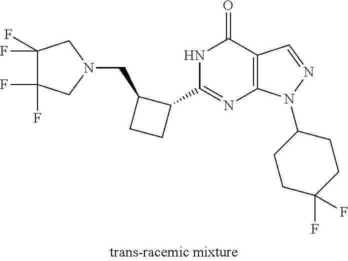

77.0 mg (0.16 mmol) of Example 4 were mixed with THF and 0.10 mL LiAlH₄ (2 M solution in THF) were added. After stirring for 30 min at reflux, the mixture was quenched with water/THF and then evaporated under reduced pressure. The residue was purified by preparative HPLC (eluent A: water+0.13% TFA, eluent B: acetonitrile). 33 mg (44%) of the product were obtained.

HPLC-MS (Method 1): Rₜ=1.59 min

MS (ESI pos): m/z=464 (M+H)⁺

### Example 29

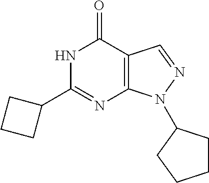

50.0 mg (0.26 mmol) of 5-amino-1-cyclopentyl-1H-pyrazole-4-carboxylic acid amide (DE 10238724) and 41.8 mg (0.51 mmol) cyclobutanecarbonitrile were mixed with 2 mL ethanol. 30.9 mg (1.29 mmol) sodium hydride (60% suspension in mineral oil) were added. The mixture was heated to 150° C. for 30 min in a microwave oven. The substance was purified by preparative HPLC (eluent A: water+0.13% TFA, eluent B: acetonitrile). 5 mg (7.5%) of the product were obtained.

HPLC-MS (Method 1): Rₜ=1.38 min

MS (ESI pos): m/z=259 (M+H)⁺

The following example was synthesized in analogy to the preparation of Example 29:

| | | starting | starting | | |
|---|---|---|---|---|---|
| | | material | material | | MS |
| Example | structure | 1 | 2 | Rₜ [min] | (ESI pos, m/z) |
| Exp. 30 | 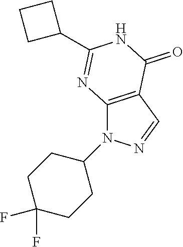 | Exp. 4A | cyclobutane- carbonitrile | 1.43 (Method 1) | 309 (M + H)⁺ |
| Exp. 31 | 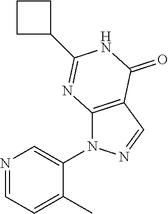 | 5-amino-1-(4- methyl- pyridin-3-yl)- 1H-pyrazole- 4-carboxylic acid amide (WO 2004/099211) | cyclobutane- carbonitrile | 0.90 (Method 1) | 282 (M + H)⁺ |

### Example 32

### Trans-Racemic Mixture

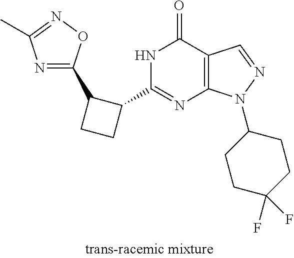

200 mg (0.57 mmol) of Example 46 were mixed with 1 mL DMF and 92.0 mg (0.57 mmol) of CDI were added. After stirring at room temperature for 6 h, 42.1 mg (0.57 mmol) of N-hydroxyacetamidine were added. After stirring for 2 h at room temperature the mixture was heated to 100° C. and stirred 16 h. The mixture was purified by preparative HPLC (eluent A: water+0.13% TFA, eluent B: acetonitrile). 160 mg (72%) of the product were obtained.

HPLC-MS (Method 1): Rₜ=1.38 min

MS (ESI pos): m/z=391 (M+H)⁺

### Example 33

### Trans-Racemic Mixture

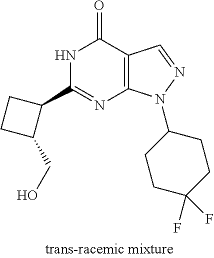

600 mg (1.70 mmol) of Example 46 were mixed with 10 mL DME and cooled to −22° C. 0.28 mL (2.55 mmol) of N-methylmorpholine and a solution of 0.29 mL (2.21 mmol) isobutyl chloroformate in DME were added. The mixture was warmed up to −5° C. and filtered. The filtrate was cooled to −15° C. and 122 mg (3.24 mmol) of sodium borohydride and two drops of water were added. The mixture was warmed to room temperature and stirred for 30 min. The solvent was evaporated under reduced pressure. The residue was extracted with ethyl acetate. The combined organic layers were dried and evaporated under reduced pressure. The residue was purified by preparative HPLC (eluent A: water+0.13% TFA, eluent B: acetonitrile). 480 mg (83%) of the product were obtained.

HPLC-MS (Method 1): Rₜ=1.28 min

MS (ESI pos): m/z=339 (M+H)⁺

### Example 34

### Cis-Racemic Mixture

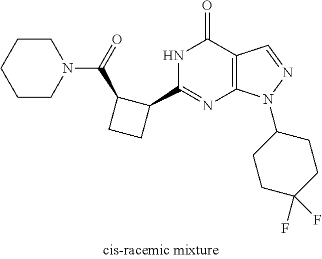

33.3 mg (0.08 mmol) of Example 12A and 49.6 mg (0.15 mmol) of cesium carbonate were mixed with 1 mL of methanol. The reaction mixture was heated to 100° C. for 30 min in a microwave oven. The mixture was cooled to room temperature. The solvent was removed under reduced pressure. The residue was purified by preparative HPLC (eluent A: water+0.13% TFA, eluent B: acetonitrile). 8 mg (25%) of the title compound were obtained as earlier eluting stereoisomer. Minor amounts of the later eluting stereoisomer Example 18 were collected separately.

HPLC-MS (Method 1): Rₜ=1.32 min

MS (ESI pos): m/z=420 (M+H)⁺

### Example 35

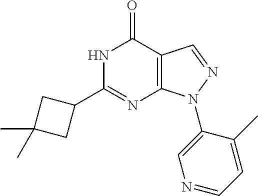

100 mg (0.46 mmol) of 5-amino-1-(4-methyl-pyridin-3-yl)-1H-pyrazole-4-carboxylic acid amide (WO 2004/099211) and 262 mg (1.84 mmol) Example 13A were mixed with 3 mL of ethanol. 55.2 mg (2.30 mmol) sodium hydride (60% suspension in mineral oil) was added. The reaction mixture was heated to 150° C. for 30 min in a microwave oven. The residue was purified by preparative HPLC (eluent A: water+0.13% TFA, eluent B: acetonitrile). 28 mg (20%) of the product were obtained.

HPLC-MS (Methode 1): Rₜ=1.10 min

MS (ESI pos): m/z=310 (M+H)⁺

The following example was synthesized in analogy to the preparation of Example 35:

| | | starting | starting | | |
|---|---|---|---|---|---|
| | | material | material | | MS |
| Example | structure | 1 | 2 | Rₜ [min] | (ESI pos, m/z) |
| Exp. 36 trans- racemic mixture | 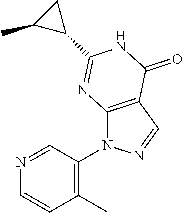 | 5-amino-1-(4- methyl- pyridin-3-yl)- 1H-pyrazole- 4-carboxylic acid amide (WO 2004/099211 | Exp. 13B | 0.91 (Method 1) | 282 (M + H)⁺ |

### Example 37

### Mixture of Stereoisomers

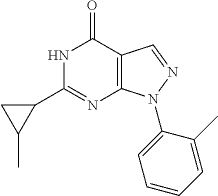

100 mg (0.46 mmol) of 5-amino-1-(2-methylphenyl)-1H-pyrazole-4-carboxamide (WO 2004/099210), 267 mg (20.8 mmol) of ethyl 2-methylcyclopropane-1-carboxylate and 157 mg (23.1 mmol) of sodium ethoxide were mixed with 25 mL ethanol and stirred at reflux overnight. The mixture was diluted with water and extracted with ethyl acetate. The combined organic layers were dried and evaporated under reduced pressure. The residue was purified by flash chromatography. 46.2 mg (36%) of the product were obtained.

HPLC-MS: Identity and purity confirmed.

The following examples were synthesized in analogy to the preparation of Example 37:

| Example | structure | starting material 1 | starting material 2 | |
|---|---|---|---|---|
| Exp. 38 mixture of stereo- isomers | 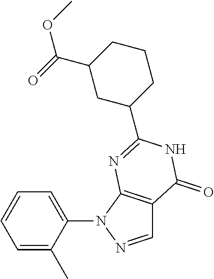 | 5-amino-1-(2- methylphenyl)-1H- pyrazole-4- carboxamide WO 2004/099210 | 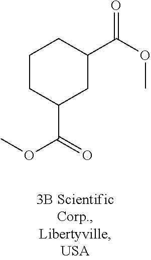 | Identity and purity confirmed by HPLC-MS |
| Exp. 39 mixture of stereo- isomers | 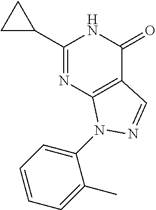 | 5-amino-1-(2- methylphenyl)-1H- pyrazole-4- carboxamide WO 2004/099210 | 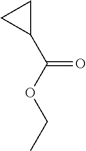 | Identity and purity confirmed by HPLC-MS |
| Exp. 40 mixture of stereo- isomers | 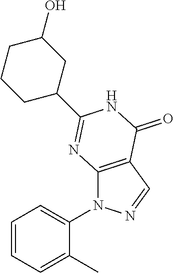 | 5-amino-1-(2- methylphenyl)-1H- pyrazole-4- carboxamide WO 2004/099210 | 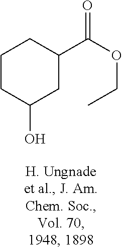 | Identity and purity confirmed by HPLC-MS and NMR |
| Exp. 41 mixture of stereo- isomers | 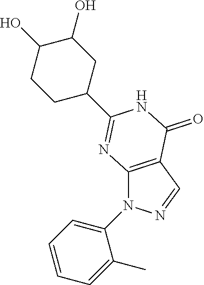 | 5-amino-1-(2- methylphenyl)-1H- pyrazole-4- carboxamide WO 2004/099210 | 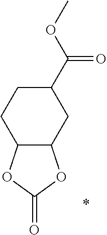 | Identity and purity confirmed by HPLC-MS and NMR |
| Exp. 42 mixture of stereo- isomers | 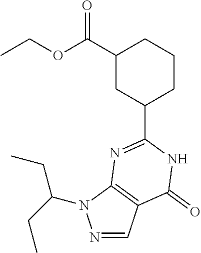 | 5-amino-1-(1- ethylpropyl)-1H- pyrazole-4- carboxamide (DE 10238722), | 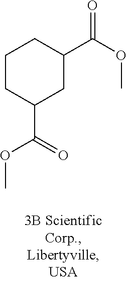 | Identity and purity confirmed by HPLC-MS |
| Exp. 43 trans- enantio- meric mixture | 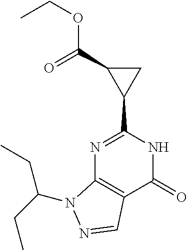 | 5-amino-1-(1- ethylpropyl)-1H- pyrazole-4- carboxamide (DE 10238722), | 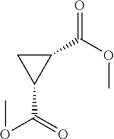 | Identity and purity confirmed by HPLC-MS |

* 2-Oxo-hexahydro-benzo[1,3]dioxole-5-carboxylic acid methyl ester can be synthesized from the corresponding diol by various
methods, e.g. with carbonic acid ditrichloromethyl ester (R. M. Burk et. al., Tetrahedron Lett., 34, 1993, 395-398).

### Example 44

### Mixture of Stereoisomers

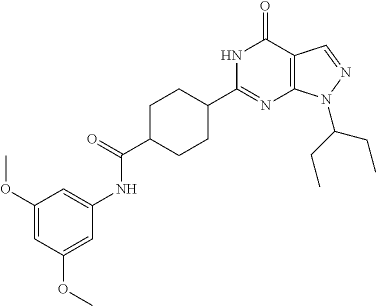

100 mg (0.30 mmol) of Example 10B, 46.1 mg (0.30 mmol) of 3,5-dimethoxyaniline, 289 mg (0.60 mmol) of HATU and 77.8 mg (0.60 mmol) of DIPEA were mixed with DMF and stirred overnight at room temperature. The reaction mixture was purified by preparative chromatography. 90.0 mg (64%) of the product were obtained.

HPLC-MS: Identity and purity confirmed

NMR: Identity confirmed

### Example 45

### Racemic Mixture, Earlier Eluting Diastereomer

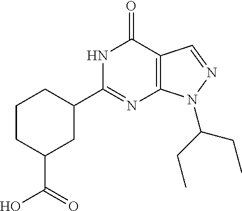

200 mg (0.56 mmol) of Example 41 and 162 mg (2.89 mmol) of potassium hydroxide were mixed with 10 mL ethanol and 1 mL water. The mixture was stirred for 12 h and the solvent was evaporated under reduced pressure. The residue was purified by preparative HPLC (eluent A: water+0.13% TFA, eluent B: acetonitrile). 40 mg (20%) of the product were obtained as the earlier eluting diastereomer.

HPLC-MS: Identity and purity confirmed

### Example 46

### Trans-Racemic Mixture

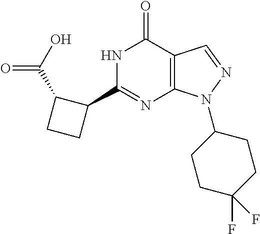

1.00 g (4.09 mmol) of Example 4A was mixed with 15 mL of absolute ethanol, 2.46 g (12.28 mmol) of Example 5A and 0.66 g (16.4 mmol) of sodium hydride (60% suspension in mineral oil) were added. The reaction mixture was heated to 140° C. for 30 min in a microwave oven. The mixture was cooled to room temperature and sodium hydroxide solution (4 M in water) was added. The solvent was removed under reduced pressure. The substance was purified by preparative HPLC (eluent A: water+0.13% TFA, eluent B: acetonitrile). 0.70 g (49%) of the product were obtained.

HPLC-MS (Method 1): Rₜ=1.24 min

MS (ESI pos): m/z=353 (M+H)⁺

### Example 47

### Trans-Racemic Mixture

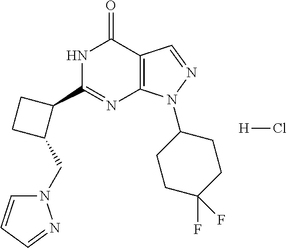

39 mg (0.58 mmol) pyrazole were mixed with 1.0 mL DMF and 19.6 mg (0.49 mmol) of sodium hydride (60% suspension in mineral oil) were added. 121 mg (0.29 mmol) of Example 6A dissolved in 1.0 mL DMF was added. The mixture was stirred for 12 h at room temperature. Dichloromethane and water were added. The organic phase was separated and the solvent was removed under reduced pressure. The residue was purified by preparative HPLC (eluent A: water+0.13% TFA, eluent B: acetonitrile). The obtained material was dissolved in dichloromethane, acidified with saturated HCl in isopropanol and evaporated to dryness to obtain 5.0 mg (4%) of the product as hydrochloride salt.

HPLC-MS (Method 1): Rₜ=1.38 min

MS (ESI pos): m/z=389 (M+H)⁺

1. A compound of formula (I) 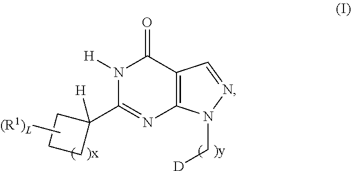
wherein
R¹ is selected independently for each R¹ from the group R^{1a} consisting of Hydrogen, fluorine, chlorine, bromine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, heterocyclyl-CO—, R¹⁰—O—, R¹⁰—O—C₁₋₃-alkyl-, (R¹⁰)₂N—, R¹⁰O—CO—, (R⁹)₂N—CO—, R¹⁰—CO—(R¹⁰)N—, R¹⁰—CO—, (R⁹)₂N—CO—(R¹⁰)N—, (R⁹)₂N—CO—O—, R¹⁰—O—CO—(R¹⁰)N—, R¹⁰—SO₂—(R¹⁰)N—, and C₁₋₆-alkyl-SO₂—,
where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, heterocyclyl-CO—, and C₁₋₆-alkyl-SO₂— may optionally be substituted independently of one another by one or more substituents selected independently of one another from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹³)₂N—C₁₋₃-alkyl-, (R¹⁰)₂N—CO—, C₃₋₆-cycloalkyl-, C₃₋₆-cycloalkyl-C₁₋₄-alkyl-, and C₁₋₆-alkyl-, preferably from the group consisting of fluorine, chlorine, bromine, OH—, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, (R¹⁰)₂N—, (R¹³)₂N—C₁₋₃-alkyl-, (R¹⁰)₂N—CO—, C₃₋₆-cycloalkyl-, and C₃₋₆-cycloalkyl-C₁₋₄-alkyl-,
L is selected from the integers 0, 1, 2 and 3,
x is selected from the integers 0, 1, 2, 3 and 4,
y is selected from the integers 0, 1 and 2,
D is selected from the group D^{1a} consisting of heterocyclyl,
wherein the above-mentioned members of the group D^{1a} may optionally be substituted by one or more substituents selected independently of one another from the group R² and/or optionally substituted by one group R³
or
D is selected from the group D^{2a} consisting of cyclopropyl, cyclobutyl, cyclopentyl, cyclohexyl, cycloheptyl cyclooctyl, cyclobutenyl, cyclopentenyl, cyclohexenyl, cycloheptenyl, cyclooctenyl, cyclopentadienyl, cyclohexadienyl, cycloheptadienyl, cyclooctadienyl, cycloheptatrienyl, cyclooctatrienyl and cyclooctatetraenyl,
wherein the above-mentioned members of the group D^{2a} may optionally be substituted by one or more substituents selected independently of one another from the group R⁴,
or
D is selected from the group D^{3a} consisting of C₁₋₈-alkyl
wherein the above-mentioned C₁₋₈-alkyl-group D^{3a} may optionally be substituted by one or more substituents selected independently of one another from the group R⁵.
or
D is selected from the group D^{4a} consisting of aryl
wherein the above-mentioned aryl group D^{4a} may optionally be substituted by one or more substituents selected independently of one another from the group consisting of R⁶. Preferred are such compounds wherein D^{4a} is substituted by not more than one R⁶.
or
D is selected from the group D^{5a} consisting of heteroaryl
wherein the above-mentioned members of the group D^{5a} may optionally be substituted by one or more substituents selected independently of one another from the group R⁶. Preferred are such compounds wherein D^{5a} is substituted by not more than one R⁶.
R² is selected from the group R^{2a} consisting of
H—, fluorine, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, C₁₋₆-alkyl-SO₂—, and oxo,
where the above-mentioned members HF₂C—, FH₂C—, F₃C—CH₂—, C₁₋₆-alkyl-(preferably C₂₋₆-alkyl), C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl, heteroaryl-C₁₋₆-alkyl-, heteroaryl-C₂₋₆-alkenyl-, heteroaryl-C₂₋₆-alkynyl-, R¹⁰—O—C₂₋₃-alkyl-, (R¹⁰)₂N—C₁₋₃-alkyl-, and C₁₋₆-alkyl-SO₂— may optionally be substituted independently of one another by one or more substituents selected independently of one another from the group consisting of
fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
and in cases in that D¹ is a heterocyclyl group with NR² as ring member, R² shall be
independently of any other R²: H—, F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, Rub-CO—, (R¹⁰)₂N—CO—, R¹⁰—CO—, R¹⁰—SO₂—, and C₁₋₆-alkyl-SO₂—,
where the above-mentioned members F₃C—CH₂—, HF₂C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, heteroaryl, heteroaryl-C₁₋₆-alkyl-, R¹⁰—O—C₁₋₃-alkyl-, and C₁₋₆-alkyl-SO₂— may optionally be substituted independently of one another by one or more substituents selected independently of one another from the group consisting of
fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and (R¹⁰)₂N—CO—,
R³ is selected from the group R^{1a} consisting of
H—, HO— and R¹⁰—O—,
R⁴ is selected from the group R^{4a} consisting of
H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
or
two substituents R^{4a} together form a C₂₋₆-alkylene bridge, wherein one or two CH₂ groups of the C₂₋₆-alkylene bridge may be replaced independently of one another by O, S, SO, SO₂, N(R¹⁰) or N—C(O)—R¹⁰ in such a way that in each case two O or S atoms or an O and an S atom are not joined together directly.
R⁵ is selected from the group R^{5a} consisting of
H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—, O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
where the above-mentioned members HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl-, and C₁₋₆-alkyl-SO₂—, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁶ is selected from the group R^{6a} consisting of
H—, fluorine, chlorine, bromine, HO—, NC—, F₃C—, HF₂C—, FH₂C—, F₃C—CH₂—, carboxy-, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, R¹⁰—O—CO—(R¹⁰)N—, R¹⁰—SO₂—(R¹⁰)N—, and C₁₋₆-alkyl-SO₂—,
where the above-mentioned members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-cycloalkyl-C₂₋₆-alkenyl-, C₃₋₇-cycloalkyl-C₂₋₆-alkynyl-, C₃₋₇-heterocyclyl-, C₃₋₇-heterocyclyl-C₁₋₆-alkyl-, C₃₋₇-heterocyclyl-C₂₋₆-alkenyl-, C₃₋₇-heterocyclyl-C₂₋₆-alkynyl-, aryl, aryl-C₁₋₆-alkyl-, aryl-C₂₋₆-alkenyl-, aryl-C₂₋₆-alkynyl-, heteroaryl-, heteroaryl-C₁₋₆-alkyl-, heteroaryl-C₂₋₆-alkenyl-, heteroaryl-C₂₋₆-alkynyl-, R¹⁰—O—C₁₋₃-alkyl-, (R¹⁰)₂N—C₁₋₃-alkyl- and C₁₋₆-alkyl-SO₂— may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
R⁹ is selected independently for each R⁹ from the group R^{9a} consisting of H—, F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, and
where the above-mentioned members F₃C—CH₂—, C₂₋₆-alkenyl-, C₂₋₆-alkinyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, heteroaryl-C₁₋₃-alkyl- and C₁₋₆-alkyl-, may optionally be substituted independently of one another by one or more substituents selected independently of one another from the group consisting of
fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl-O— and C₁₋₆-alkyl-,
R¹⁰ is selected independently for each R¹⁰ from the group R^{10a} consisting of H— (but not in case is part of a group being selected from R¹⁰O—CO—, R¹⁰—SO₂— or R¹⁰—CO—), F₃C—CH₂—, C₁₋₆-alkyl-, C₂₋₆-alkenyl-, C₃₋₇-cycloalkyl-, C₃₋₇-cycloalkyl-C₁₋₃-alkyl-, C₃₋₇-heterocyclyl-, C₃₋₇-heterocyclyl-C₁₋₆-alkyl-, aryl, aryl-C₁₋₃-alkyl-, heteroaryl, and heteroaryl-C₁₋₃-alkyl-,
and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —S—, —NH—, —N(C₃₋₆-cycloalkyl)-, —N(C₃₋₆-cycloalkyl-C₁₋₄-alkyl)- or —N(C₁₋₄-alkyl)- and
where the above-mentioned members may optionally be substituted independently of one another by one or more substituents selected from the group consisting of
fluorine, chlorine, bromine, HO—, NC—, O₂N—, F₃C—, HF₂C—, FH₂C—, F₃C—CH₂—, HO—C₁₋₆-alkyl-, CH₃—O—C₁₋₆-alkyl-, C₁₋₆-alkyl- and C₁₋₆-alkyl-O—,
and pharmaceutically acceptable salts thereof.

2. A compound according to claim 1, wherein
D is selected from the group D^{1b} consisting of heterocyclyl,
defined by any of formulas I.1 or I.2 or I.3: 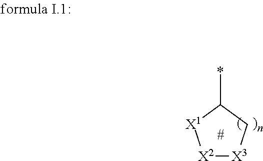
with
n=1, 2, 3;
# meaning that the ring is not aromatic while for n=1, one bond within the ring system optionally may be a double bond and for n=2 or 3 one bond or two bonds within the ring system optionally may be (a) double bond(s), For each occasion the double bond preferably is a C—C double bond. Preferably the ring system is saturated,
X¹, X², X³, independently from each other being CH₂, CHR², CHR³, C(R²)₂, CR²R³, O, NH, NR², or S(O), with r=0, 1, 2, whereby at least one of X¹, X², X³ is O, NH, NR² or S(O)ᵣ, whereby the substituents R² and R³ are selected independently of each other;
The * represents the point of attachment to the nitrogen atom of the pyrazolo ring of formula I; 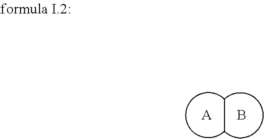
with
A being the ring system of formula I.1;
B being a 3, 4, 5 or 6 membered second ring systems that is anellated to A and that beside the two atoms and one bond it shares with A consists only of carbon atoms and that may be saturated, partially saturated or aromatic, the ring system of formula I.2 may optionally be substituted by one or more substituents selected independently of one another from the group R² and/or optionally substituted by one group R³ and whereby the two ring atoms that are shared by the two ring systems A and B both may be C-atoms, both may be N-atoms or one may be a C- and the other one may be a N-atom. Preferred are two C-atoms, or one C- and one N-atom, and more preferred are two C-atoms. The shared bond may be a single bond or a double bond; 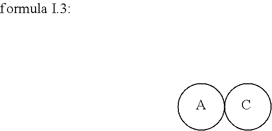
with
A, being the ring system of formula I.1;
C being a 3, 4, 5 or 6 membered second ring systems that is spiro fused to A and that beside the one atoms it shares with A consists only of carbon atoms and that may be saturated, partially saturated or aromatic whereby the ring system of formula I.3 may optionally be substituted by one or more substituents selected independently of one another from the group R² and/or optionally substituted by one group R³ and whereby the ring atom that is shared by the two ring systems A and C is a C-atom,
or
D is selected from the group D^{2b} consisting of cyclopropyl, cyclobutyl, cyclopentyl, cyclohexyl, cycloheptyl, cyclopentenyl and cyclohexenyl, wherein the above-mentioned members of the group D^{2b} may optionally be substituted by one or more substituents selected independently of one another from the group R⁴,
or
D is selected from the group D^{3b} consisting of C₁₋₆-alkyl
wherein the above-mentioned C₁₋₆-alkyl-group may optionally be substituted by one or more substituents selected independently of one another from the group R⁵,
or
D is selected from the group D^{4b} consisting of phenyl
wherein the above-mentioned phenyl group may optionally be substituted by one or more substituents selected independently of one another from the group consisting of R⁶,
or
D is selected from the group D^{5b} consisting of pyridyl
wherein the above-mentioned pyridyl group may optionally be substituted by one or more substituents selected independently of one another from the group R⁶.

3. A compound according to claim 1, wherein
R¹ is selected independently for each R¹ from the group R^{1b} consisting of Hydrogen, fluorine, F₃C—, F₃C—CH₂—, C₁₋₆-alkyl-, C₁₋₃-alkynyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, heterocyclyl-CO—, R¹⁰—O—, R¹⁰—O—C₁₋₃-alkyl-, (R¹⁰)₂N—, R¹⁰O—CO—, (R⁹)₂N—CO—, R¹⁰—CO—(R¹⁰)N—, (R⁹)₂N—CO—(R¹⁰)N—, (R⁹)₂N—CO—O—, and R¹⁰—O—CO—(R¹⁰)N—,
where the above-mentioned, members F₃C—CH₂—, C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, heterocyclyl-CO—, and R¹⁰—O—C₁₋₃-alkyl-, may optionally be substituted independently of one another by one or more substituents selected independently of one another from the group consisting of fluorine, HO—, F₃C—, HF₂C—, FH₂C—, F₃C—CH₂—, C₁₋₆-alkyl-O—, (R¹⁰)₂N—, (R¹⁰)₂N—C₁₋₃-alkyl-, (R¹⁰)₂N—CO—, C₃₋₆-cycloalkyl-, C₃₋₆-cycloalkyl-C₁₋₄-alkyl- and C₁₋₆-alkyl-.

4. A compound according to claim 1, wherein
R² is selected from the group R^{2b} consisting of
H—, fluorine, F₃C—, HF₂C—, FH₂C—, F₃C—CH₂—, C₁₋₆-alkyl-, (R¹⁰)₂N—CO—, R¹⁰—CO—(R¹⁰)N—,
where the above-mentioned members may optionally be substituted independently of one another by one or more substituents selected from the group consisting of
fluorine, chlorine, bromine, NC—, O₂N—, F₃C—, HF₂C—, FH₂C—, F₃C—CH₂—, HO—C₁₋₆-alkyl-, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,
and in cases where D is a heterocyclyl group with NR² as ring member, R² shall be
independently of any other R²: H—, F₃C—CH₂—, HF₂C—CH₂—, C₁₋₆-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl, heteroaryl-C₁₋₆-alkyl-, R¹⁰—O—C₁₋₃-alkyl-, Rub-CO—, (R¹⁰)₂N—CO—, R¹⁰—CO—, and C₁₋₆-alkyl-SO₂—,
where the above-mentioned members may optionally be substituted independently of one another by one or more substituents selected from the group consisting of
fluorine, HO—, NC—, O₂N—, F₃C—, HF₂C—, FH₂C—, F₃C—CH₂—, HO—C₁₋₆-alkyl-, R¹⁰—O—C₁₋₆-alkyl-, C₁₋₆-alkyl-, R¹⁰—O—, (R¹⁰)₂N—, (R¹⁰)₂N—C₁₋₃-alkyl-, and (R¹⁰)₂N—CO—.

5. A compound according to claim 1, wherein
R³ is selected from the group R^{3b} consisting of
H—, hydroxy, C₁₋₆-alkyl-O—, whereby C₁₋₆-alkyl-O— may optionally be substituted by one or more fluorine and/or one HO—.

6. A compound according to claim 1, wherein
R⁴ is selected from the group R^{4b} consisting of
H—, fluorine, NC—, F₃C—, HF₂C—, FH₂C—, F₃C—CH₂—, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)₂N—, (R¹⁰)₂N—C₁₋₃-alkyl-, Rub-CO—, (R¹⁰)₂N—CO—, R¹⁰—CO—(R¹⁰)N—, R¹⁰—CO—, (R¹⁰)₂N—CO—(R¹⁰)N— and R¹⁰—O—CO—(R¹⁰)N—,
where the above-mentioned members F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl- and (R¹⁰)₂N—C₁₋₃-alkyl-, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—.

7. A compound according to claim 1, wherein
R⁵ is selected from the group R^{5b} consisting of
H—, fluorine, NC—, F₃C—, HF₂C—, FH₂C—, F₃C—CH₂—, C₁₋₆-alkyl-, C₂₋₆-alkenyl-, C₂₋₆-alkynyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)₂N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, and R¹⁰—O—CO—(R¹⁰)N—,
where the above-mentioned, members HF₂C—, FH₂C—, F₃C—CH₂—, C₁₋₆-alkyl-, C₂₋₆-alkenyl-, C₂₋₆-alkynyl-, C₁₋₆-alkyl-S—C₁₋₃-alkyl-, C₃₋₇-cycloalkyl-, C₃-7-cycloalkyl-C₁₋₆-alkyl-, C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl-, and (R¹⁰)₂N—C₁₋₃-alkyl- may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,

8. A compound according to claim 1, wherein
R⁶ is selected from the group R^{6b} consisting of
H—, fluorine, chlorine, bromine, HO—, NC—, F₃C—, HF₂C—, FH₂C—, F₃C—CH₂—, carboxy-, C₁₋₆-alkyl-, C₁₋₆-alkyl-S—, C₁₋₆-alkyl-S—C₁₋₃-alkyl-, C₃₋₇-cycloalkyl-, C₃₋₇-cycloalkyl-C₁₋₆-alkyl C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—, R¹⁰—O—C₁₋₃-alkyl-, (R¹⁰)²N—, (R¹⁰)₂N—C₁₋₃-alkyl-, R¹⁰O—CO—, (R¹⁰)₂N—CO—, R¹⁰—CO—(R¹⁰)N—, R¹⁰—CO—, (R¹⁰)₂N—CO—(R¹⁰)N—, and R¹⁰—O—CO—(R¹⁰)N—,
where the above-mentioned, members HF₂C—, FH₂C—, F₃C—CH₂—, C₁₋₆-alkyl-, C₁₋₆-alkyl-S—C₁₋₃-alkyl-, C₃₋₇-cycloalkyl-, C₃₋₇-cycloalkyl-C₁₋₆-alkyl C₃₋₇-heterocyclyl-, C₃₋₇-heterocyclyl-C₁₋₆-alkyl-, aryl, aryl-C₁₋₆-alkyl-, heteroaryl-, heteroaryl-C₁₋₆-alkyl-, R¹⁰—O—C₁₋₃-alkyl- and (R¹⁰)₂N—C₁₋₃-alkyl-, may optionally be substituted independently of one another by one or more substituents selected from the group consisting of fluorine, chlorine, bromine, NC—, O₂N—, F₃C—, HF₂C—, FH₂C—, F₃C—CH₂—, HO—C₁₋₆-alkyl-, C₁₋₆-alkyl-O—, C₁₋₆-alkyl-O—C₁₋₆-alkyl-, C₁₋₆-alkyl-, (R¹⁰)₂N—, (R¹⁰)₂N—C₁₋₃-alkyl-, and (R¹⁰)₂N—CO—,

9. A compound according to claim 1, wherein
R⁹ is selected independently for each R⁹ from the group R^{9b} consisting of H—, C₁₋₆-alkyl-, C₂₋₆-alkinyl-, C₃₋₇-cycloalkyl-, C₃₋₇-cycloalkyl-C₁₋₃-alkyl-, aryl, aryl-C₁₋₃-alkyl-, heteroaryl, and heteroaryl-C₁₋₃-alkyl-,
where the above-mentioned members C₁₋₆-alkyl-, C₂₋₆-alkinyl-, C₃₋₇-cycloalkyl-, C₃₋₇-cycloalkyl-C₁₋₃-alkyl-, aryl, aryl-C₁₋₃-alkyl-, heteroaryl, and heteroaryl-C₁₋₃-alkyl- may optionally be substituted independently of one another by one or more substituents selected independently from one another from the group consisting of
fluorine, NC—, F₃C—, HF₂C—, FH₂C—, F₃C—CH₂—, CH₃—O—C₁₋₆-alkyl-, C₁₋₆-alkyl-O— and C₁₋₆-alkyl-.

10. A compound according to claim 1, wherein
R¹⁰ is selected independently for each R¹⁰ from the group R^{10b} consisting of H— (but not in case is part of a group being selected from R¹⁰O—CO—, R¹⁰—SO₂— or R¹⁰—CO—), C₁₋₆-alkyl-, C₃₋₇-cycloalkyl-, C₃₋₇-cycloalkyl-C₁₋₃-alkyl-, aryl and heteroaryl,
and in case where two R¹⁰ groups both are bound to the same nitrogen atom they may together with said nitrogen atom form a 3 to 12 membered heterocyclyl ring, and wherein one of the —CH₂-groups of the heterocyclic ring formed may be replaced by —O—, —NH—, —N(C₃₋₆-cycloalkyl)-, —N(C₃₋₆-cycloalkyl-C₁₋₄-alkyl)- or —N(C₁₋₄-alkyl)- and
where the above-mentioned members may optionally be substituted independently of one another by one or more substituents selected from the group consisting of
fluorine, NC—, F₃C—, HF₂C—, FH₂C—, F₃C—CH₂—, CH₃—O—C₁₋₆-alkyl-, C₁₋₆-alkyl-, and C₁₋₆-alkyl-O—.

11. A compound according to claim 1, wherein
D is selected from the group consisting of cyclopentyl, cyclohexyl, 3-pentyl-, phenyl, tetrahydropyranyl, tetrahydrofuranyl and pyridyl, wherein the above-mentioned cyclopentyl, cyclohexyl, 3-pentyl-, phenyl, and pyridyl groups may optionally be substituted by one or more substituents selected independently of one another from the group consisting of fluorine, chlorine or methyl and
wherein the above-mentioned tetrahydropyranyl and tetrahydrofuranyl groups may optionally be substituted by one or more substituents selected independently of one another from the group consisting of methyl.
L is selected from the integers 0, 1 and 2,
x is selected from the integers 0, 1, 2 and 3,
y is 0,
and
R¹ is selected independently for each R¹ from the group R^{1d} consisting of C₁₋₃-alkyl-, C₁₋₃-alkyl-O—CO—, HO—CO—, (C₁₋₆-alkyl)₂N—CO—, oxadiazolyl, oxazolyl, isoxazolyl, triazolyl, thiazolyl, thienyl, pyrrolyl, furanyl, pyrazolyl, pyridyl, pyridazinyl, pyrimidinyl, oxadiazolyl-C₁₋₃-alkyl-, oxazolyl-C₁₋₃-alkyl-, isoxazolyl-C₁₋₃-alkyl-, triazolyl-C₁₋₃-alkyl-, thiazolyl-C₁₋₃-alkyl-, pyrrolyl-C₁₋₃-alkyl-, furanyl-C₁₋₃-alkyl-, pyrazolyl-C₁₋₃-alkyl-, pyridyl-C₁₋₃-alkyl-, pyridazinyl-C₁₋₃-alkyl-, pyrimidinyl-C₁₋₃-alkyl-, pyrazolyl-C₁₋₃-alkyl-, 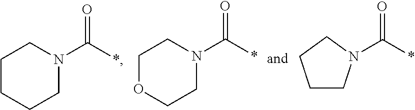
where the above-mentioned, members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl-.

12. A compound according to claim 1, wherein
D is selected from the group consisting of cyclopentyl, cyclohexyl, 3-pentyl-, phenyl and pyridyl,
wherein the above-mentioned groups may optionally be substituted by one or more substituents selected independently of one another from the group consisting of fluorine, chlorine or methyl.
L is 1,
x is selected from the integers 1 and 2,
y is 0,
and
R¹ is selected independently for each R^{1e} from the group consisting of C₁₋₃-alkyl-, (C₁₋₆-alkyl)₂N—CO—, oxadiazolyl, pyrazolyl-methyl-, 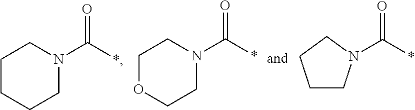
where the above-mentioned, members may optionally be substituted independently of one another by one or more substituents selected independently of one another from the group consisting of fluorine, F₃C—, HF₂C—, FH₂C—, F₃C—CH₂—, C₁₋₃-alkyl-O— and C₁₋₃-alkyl.

13. A compound according to claim 1, wherein
D is selected from the group consisting of cyclopentyl, cyclohexyl and pyridyl, wherein the above-mentioned groups may optionally be substituted by one or more substituents selected independently of one another from the group consisting of fluorine, chlorine or methyl.

14. A compound according to claim 1 with the provision that
if D is oxetanyl, which is bound via the carbon atom next to the oxygen of the oxetanyl, there is no substituent attached to said carbon atom via an integral —CH₂-group.

15. A compound according to claim 1, wherein the compound is selected from the group of

| No. | Compound |
|---|---|
| 1 | 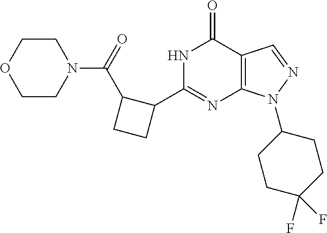 |
| 2 | 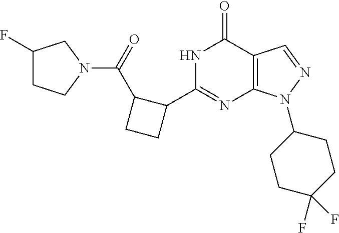 |
| 3 | 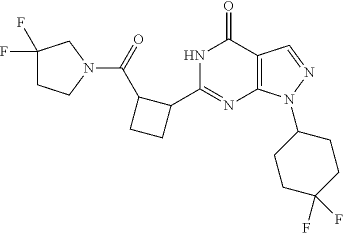 |
| 4 | 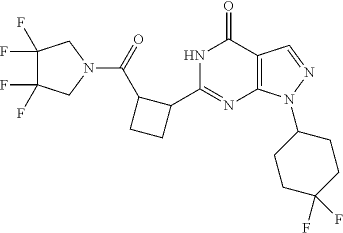 |
| 5 | 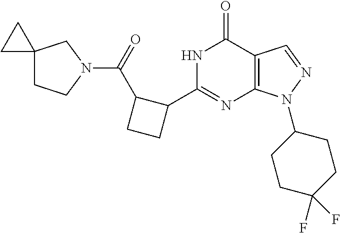 |
| 6 | 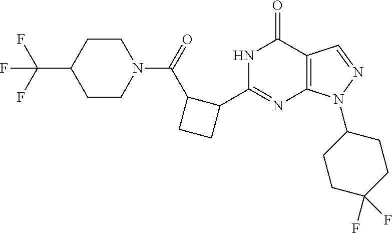 |
| 7 | 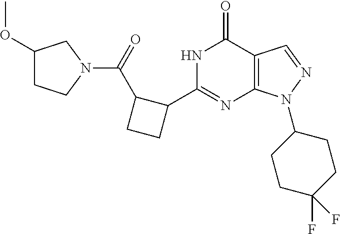 |
| 8 | 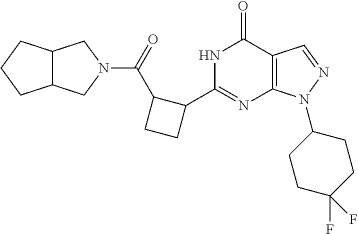 |
| 9 | 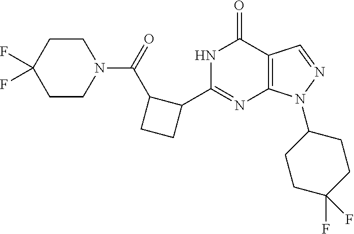 |
| 10 | 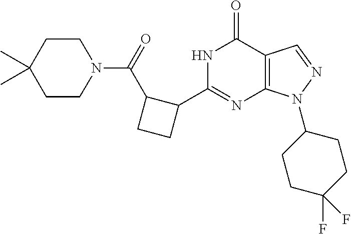 |
| 11 | 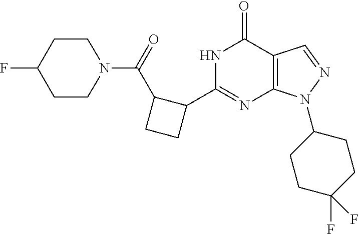 |
| 12 | 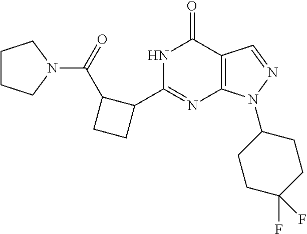 |
| 13 | 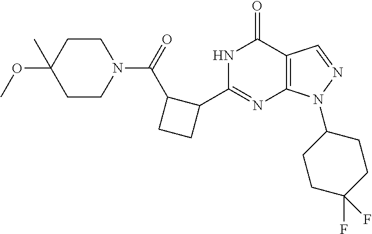 |
| 14 | 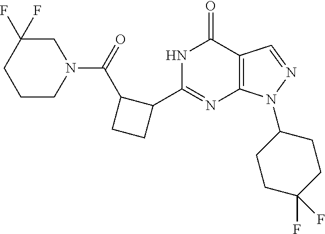 |
| 15 | 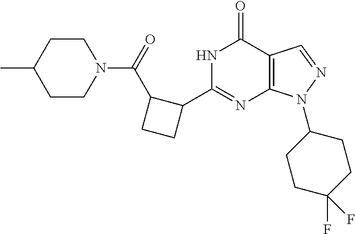 |
| 16 | 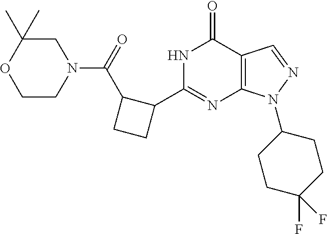 |
| 17 | 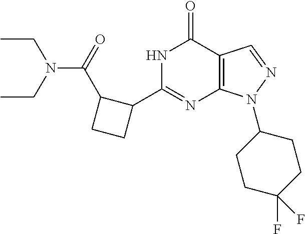 |
| 18 | 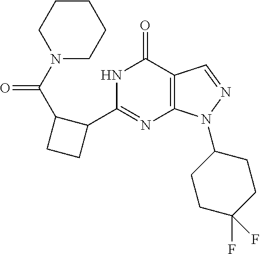 |
| 19 | 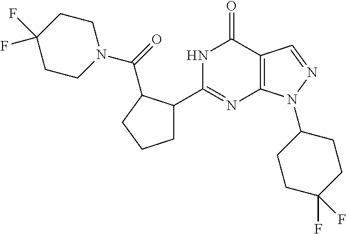 |
| 20 | 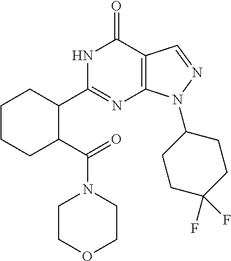 |
| 21 | 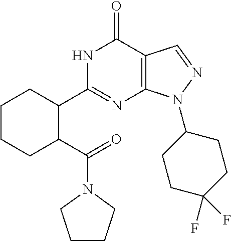 |
| 22 | 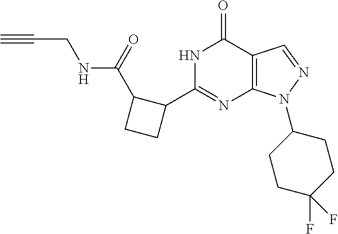 |
| 23 | 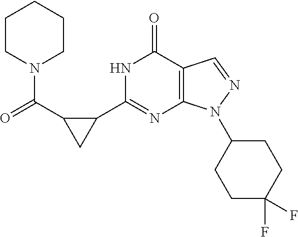 |
| 24 | 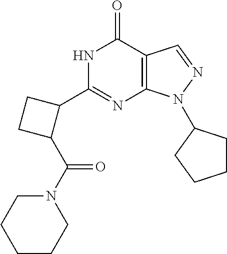 |
| 25 | 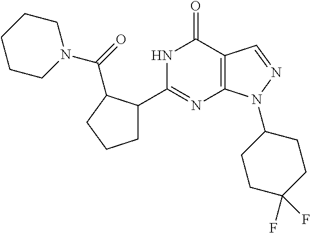 |
| 26 | 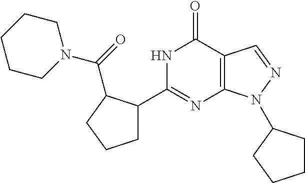 |
| 27 | 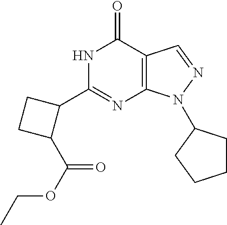 |
| 28 | 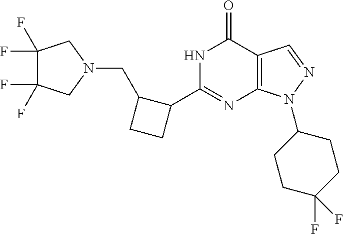 |
| 29 | 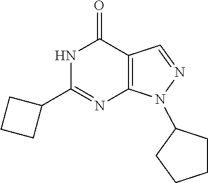 |
| 30 | 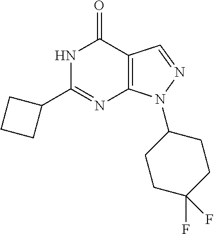 |
| 31 | 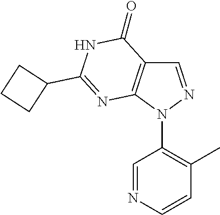 |
| 32 | 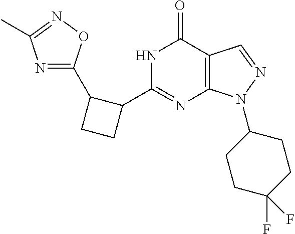 |
| 33 | 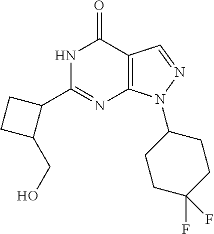 |
| 34 | 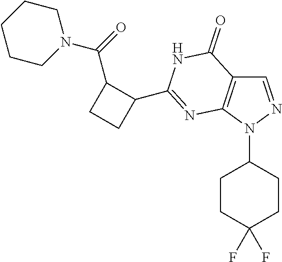 |
| 35 | 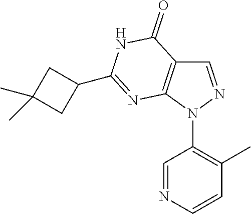 |
| 36 | 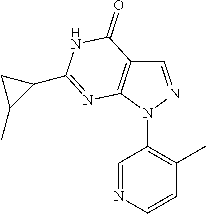 |
| 37 | 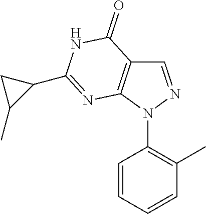 |
| 38 | 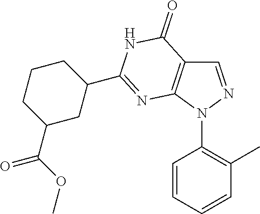 |
| 39 | 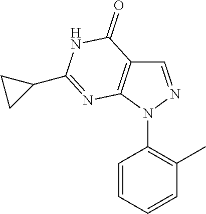 |
| 40 | 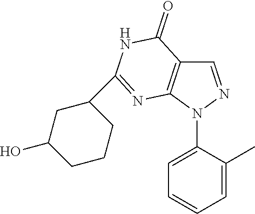 |
| 41 | 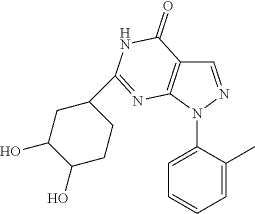 |
| 42 | 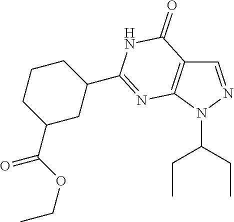 |
| 43 | 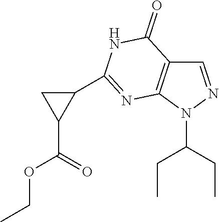 |
| 44 | 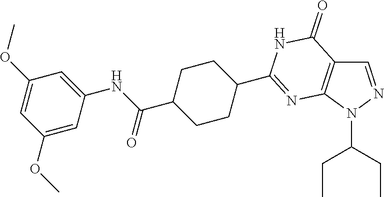 |
| 45 | 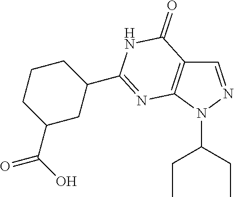 |
| 46 | 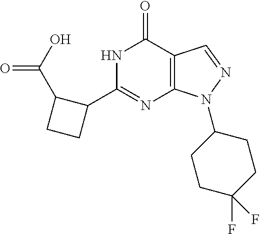 |
| 47 | 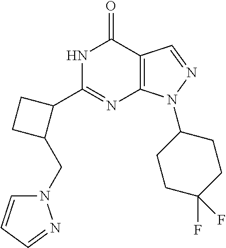 |

and salts thereof, preferably pharmaceutically acceptable salts thereof.

16. Method for the treatment of a CNS disease comprising administering to a patient a compound according to claim 1.

17. Method according to claim 16, wherein the treatment is a CNS disease, the treatment of which is accessible by the inhibition of PDE9.

18. Method for the treatment of a disease that is accessible by the inhibition of PDE9 comprising administering to a patient a compound according to claim 1.

19. Method for the treatment, amelioration or prevention of a disease or condition selected from the group of cognitive impairment being related to perception, concentration, cognition, learning and memory comprising administering to a patient a compound according to claim 1.

20. Method according to claim 19, wherein the medicament is for the treatment, amelioration or prevention of a disease or condition selected from the group of cognitive impairment being related to age-associated learning and memory impairments, age-associated memory losses, vascular dementia, craniocerebral trauma, stroke, dementia occurring after strokes (post stroke dementia), post-traumatic dementia, general concentration impairments, concentration impairments in children with learning and memory problems, Alzheimer's disease, Lewy body dementia, dementia with degeneration of the frontal lobes, including Pick's syndrome, Parkinson's disease, progressive nuclear palsy, dementia with corticobasal degeneration, amyotropic lateral sclerosis (ALS), Huntington's disease, multiple sclerosis, thalamic degeneration, Creutzfeld-Jacob dementia, HIV dementia, schizophrenia with dementia and Korsakoff's psychosis.

21. Method for the treatment of Alzheimer's disease comprising administering to a patient a compound according to claim 1.

22. Method for the treatment of cognitive impairment which is due to Alzheimer's disease comprising administering to a patient a compound according to claim 1.

23. Method for the treatment of a disease or condition selected from the group of sleep disorders, bipolar disorder, metabolic syndrome, obesity, diabetes mellitus, hyperglycemia, dyslipidemia, impaired glucose tolerance, and a disease of the testes, brain, small intestine, skeletal muscle, heart, lung, thymus and spleen comprising administering to a patient a compound according to claim 1.

24. Pharmaceutical composition comprising a compound according to claim 1 and a pharmaceutical carrier.

25. Combination of a compound according to claim 1 with another active agent, for example another active agent for the treatment of Alzheimer's disease.
